(19) United States
(12) Patent Application Publication
(10) Pub. No.: US 2016/0355836 A1
(43) Pub. Date: Dec. 8, 2016

(54) MODIFIED PLANTS

(71) Applicant: ZHEJIANG UNIVERSITY, Hangzhou (CN)

(72) Inventors: Ping Wu, Hangzhou (CN); Jieyu Chen, Hangzhou (CN)

(21) Appl. No.: 15/031,993

(22) PCT Filed: Feb. 20, 2014
(86) PCT No.: PCT/CN2014/072289

§ 371 Date: Apr. 25, 2016

(30) Foreign Application Priority Data
Oct. 25, 2013 (CN) 201310513975.X

(51) Int. Cl. C12N 15/82; C07K 14/415; G01N 33/68

(52) U.S. Cl. CPC C12N 15/8261; G01N 33/68; C07K 14/415; G01N 2333/415; G01N 2440/14

(57) ABSTRACT

Disclosed are improved plants that have increased yield. The plants show increased yield under low phosphate conditions and therefore require less fertilizer. The plants are characterised by expression of a mutant phosphate transporter gene.

The essential plant macronutrient phosphate (Pi) has drawn increasing attention because heavy application of P-fertilizers in agriculture to sustain higher yield results in serious environmental problems, and thus non-renewable Pi resource is predicted to be exhausted within 70 to 200 years (1, 2). Improving Pi use efficiency of plants is thus an important goal for sustainable agricultural production.

Phosphorus is an essential macronutrient for plant growth and development. Pi deficient plants generally turn dark green and appear stunted. Plants acquire Pi directly from their environment by active absorption into the epidermal and cortical cells of the root via Pi transporters. After entry into the root cortical cells, Pi must eventually be loaded into the apoplastic space of the xylem, transported to the shoot and then redistributed within the plant via Pi transporters. As a constituent of nucleic acids, phospholipids and cellular metabolites, living cells require millimolar amounts of Pi. However, most soil Pi is immobile and the Pi concentration available to roots is in micromolar quantities. Too much Pi uptake does however lead to the Pi toxicity syndrome.

To coordinate plant growth with the limited Pi availability, high affinity Pi transporters have evolved to enable increased Pi acquisition from soils. High-affinity plant Pi transporters in plants were originally identified by sequence similarity with the high-affinity transporter of yeast, PHO84. Genes encoding some of these transporters are able to complement pho84 yeast mutants. These proteins belong to the PHOSPHATE TRANSPORTER1 (PHT1) family of Pi/H+ symporters. Nine PHT1 genes have been identified in Arabidopsis (Arabidopsis thaliana), and 13 PHT1 genes have been identified in rice (Oryza sativa). Following protein synthesis, these plasma membrane (PM) proteins are initially targeted to the endoplasmic reticulum (ER), after which they require various trafficking steps to reach their final destination.

Another regulator of the Pi signalling pathway is the PHOSPHATE TRANSPORTER TRAFFIC FACILITATOR1 (PHF1) (3). This gene encodes a protein located in the ER that is required for the correct targeting of the PHTprotein from the ER to the PM. Overexpression of OsPHF1 results in an increase of Pi accumulation at high Pi concentration in transgenic rice. In Arabidopsis however, overexpression of AtPHF1 did not lead to significantly increased uptake of Pi (4, 5). Thus, despite increased PHF activity resulting in translocation of PHT from the ER to the PM, this did not lead to increased Pi uptake in Arabidopsis.

In Arabidopsis, mutants of AtPHT1;1 which have mutations in a number of phosphorylation sites mimicking unphosphorylated or phosphorylated residues respectively have been studied. Wild type and mutant versions of AtPHT1;1 were expressed in Arabidopsis. It has been suggested that phosphorylation events at the C-terminus of PHT1;1 are involved in preventing exit of PHT1:1 from the ER. On the other hand, it was shown that the non-phosphorylatable mutants of AtPHT1;1 do not affect the degradation and stability process of PHT1;1 in the PM (5). Phosphorylation sites were also identified in the AtPHT1;1 homolog in rice, OsPHT1;8 (OsPT8) (4).

OsPT8 is involved in phosphate homeostasis in rice. Increased gene expression of OsPT8 in rice enhanced Pi uptake and overexpressing plants showed a reduction in growth (9). Thus, it has also been demonstrated that increased Pi uptake does not necessarily result in an advantageous phenotype: overexpression of OsPT2 and OsPT8 causes excessive shoot Pi accumulation and results in a Pi toxicity phenotype, similar to the overexpression of OsPHR2 (9).

The present invention is aimed at providing plants with an advantageous phenotype of increased Pi uptake and increased yield at low external Pi concentrations. Such plants therefore require less P-fertilizers to sustain higher yield results and address the need for a reduction of P-fertilizers in agriculture.

### DESCRIPTION OF THE FIGURES

The invention is described in the following non-limiting figures.

FIG. 1. CK2β3 directly interacts with PT and is necessary for CKα3 interaction with PT. (A) Yeast two-hybrid assay showing that only CK2β3 interacted with PT2 and PT8 in yeast cells among the four CK2 subunits (a2, a3, β1 and β3). EV, empty vector; SD/LW, -Leu-Trp; SD/LWHA, -Leu-Trp-His-Ade; +Positive control (NubI). (B) In vivo co-immunoprecipitation assays with the highly conserved carboxy terminal peptides of PT2&8(PT2-CT&PT8-CT) CK2α3 and CK2β3. Protein extracts from agro-infiltrated tobacco plants expressing PT2-CT-GFP or PT8-CT-GFP, and CK2α3-FLAG or CK2β3-MYC. (Input) were immunoprecipitated (IP) with anti-GFP and the immunoblots were developed using tag-specific antibodies. (C) CK2β3 is necessary for the interaction of CK2α3 with PT2-CT and PT8-CT in a yeast three-hybrid assay (Y3H). SD/LMW, -Leu-Met-Trp; SD/LMWH, -Leu-Met-Trp-His; EV, empty vector. (D) In vivo co-immunoprecipitation of PT8-CT, CK2α3 and CK2β3. Protein extracts from agro-infiltrated tobacco plants expressing GFP (control), CK2α3-FLAG, PT8-CT-GFP and CK2β3-MYC in the indicated combinations (Input) were immunoprecipitated (IP) with anti-GFP and immunoblots were developed using tag-specific antibodies. (E) Confocal analysis of PT8-GFP (PT8p-PT8-GFP) subcellular localization in the epidermis cells of rice roots of 7-d-old transgenic plants harbouring the PT8-GFP construct either alone (left), or simultaneously with CK2α3 (middle) or CK2β3 overexpression constructs (right). Bar=20 μm.

FIG. 2. CK2α3-mediated phosphorylation of PT8 and CK2α3 interacts with CK2β3 are dependent on cellular Pi status and impairs interaction of PT8 with PHF1. (A) Phosphorylation of PT8 by CK2α3 in vivo. Lower mobility bands were observed in the wild type (wt) and CK2α3-overexpression (Ox α3) plants, but not in CK2α3-knockdown (Ria3) plants (upper). These bands are sensitive to λ-phosphatase treatment (λ-PPase) (lower). The immunoblots were developed with anti-PT8 in Phostag SDS-PAGE. (B) Cellular Pi-dependent phosphorylation and λ-PPase sensitivity of CK2β3. Non-phosphorylatable CK2β3 was also reduced on −P. Comassie brilliant blue (CBB) staining was used as loading control of total proteins. (C) Cellular Pi sensitivity of the interaction between CK2β3 with CK2α3. Proteins of β3-FLAG was purified from respective transgenic plants grown under +Pi or −Pi conditions, and GST-a3 was purified in E. coli, then subjected to GSTPull-down assays. The experiment was performed using a similar amount of CK2β3 in the +P and −P extracts (50 ng). β3-FLAG/GST-α3 proteins were detected by immunoblot using anti-GST or anti-FALG antibody. Purified GST-α3 and β3-FLAG proteins were loaded as the input lane. (D) PHF1 doesn't interact with phosphorylated PT8 in vitro based on a pull-down assay. Shown is a western blotting of gel containing resolved affinity-purified bindingreactions that contained PHF1-MYC (top panel), GST (negative control), GST-PT8-CTS517 and GST-PT8-CTS517A (bottom). The CK2α3-mediated phosphorylated PT8-CTS517 is indicated by the signal developed after treatment with anti phosphoserine antibody (middle).

FIG. 3. Phosphorylation-dependent recycling/degradation process of PT8 at PM. (A) Subcellular localization of PT8S517-GFP (PT8p-PT8S517-GFP) and PT8S517A-GFP (PT8p-PT8S517A-GFP) in the root epidermis cells of rice seedlings grown under Pi-supplied (+P: 200 μM) and Pi-starvation (−P) conditions. The GFP images were examined after CHX (50 μM) treatment for 60 minutes using confocal microscope. Bar=10 mm. The stabilization of PT8S517A at PM level under wide Pi regimes are shown in FIG. 5. (B) A model for ER-exit of Pi transporter and recycling/degradation process at PM under the control of PHF1 and active CK2α3β3 holoenzyme as a function of cellular Pi status. At high Pi level, the phosphorylated CK2β3 interacted with CK2 α3 as an active holoenzyme phosphorylates PT and consequently inhibits interaction of PHF1 with phosphorylated PT resulting in ER-retention of PT. At low Pi level, the phosphorylation of CK2β3 is inhibited, and PHF1 interacts with non-phosphorylable PT in the meantime for efficient transition of PT from ER to PM and a recycling process at PM. Non-phosphorylatable CK2β3 is prone to be degraded on −P in lytic vacuoles. The arrow line represents enhanced effect and the arrow dashed line represents reduced effect. TGN, Trans-Golgi network; ER, endoplasmic reticulum and PM, plasma membrane.

FIG. 4. Plants with nonphosphorylatable PT8 (PT8S517A) display improved performance under low Pi regimes. (A) Growth performances of the rice cultivar XS134 (japonica cv.) and two independent transgenic lines (T2) harboring PT8S517A in a solution culture experiment with 50 and 10 μM Pi for 45 days. Bar=10 cm. (B) Dry weight of shoots and roots of the plants shown in (A). (C, and D) Cellular Pi concentrations (C) and total P (D) in shoots of the plants shown in (A). Error bars represent s.d. (n=6). Data significantly different from the corresponding the wild type controls (XS134) are indicated (** P<0.01; Student's t test). FW, fresh weight. (E and F) Growth performance (E) and yield (F) shown in one replication of XS134 and two lines of transgenic plants with PT8S517A in a low-P soil without application of P-fertilizer. N and K were applied at usual levels (450 kg urea/ha; 300 kgKCl/ha). The plants were transplanted as 4×5 plants with 25 cm×25 cm in three replications randomly arranged.

FIG. 5. Non-phosphorylatable PT8 (PT8S517) is more stabilized at PM-enriched protein. (a) PT8 protein levels in PM-enriched protein fraction in roots of the 15-d-old control (wt: XS134, japonica cv.) and transgenic plants with single copy of nonphosphorylatable PT8S517A-1 or of wt PTS517-1 after CHX treatment at 50 μM for 60 min under different Pi levels. PT accumulation was detected by Western blotting developed with anti-PT8 antibody. Comassie brilliant blue (CBB) staining was used as loading control of PM-enriched proteins. wt, the wild type XS134. (b) Quantification of the results shown in (a). Relative PT protein (fold) is the ratio of the PT8S517A signal under the given Pi level to the PT8S517 signal. Values represent mean±s.d. (n=3) (c) The relative amount of PT protein of the results shown in (a) under different Pi levels was calculated and plotted on a semilog graph. Values represent mean±s.d. (n=3).

FIG. 6. Alignment of OsPHT1;8 (OSPT8) with othologs. Orthologs in other monocot (above line) and dicot (below line) plants. The conserved S517 site in the orthologs is shown. Sequences as shown starting with the top sequence:

SEQ NO:5: Brachypodium distachyon (version XP_003573982.1 GI:357146410)
SEQ NO:7: AAO72437.1 Hordeum vulgare subsp. vulgare (version AAO72437.1 GI:29367131)
SEQ NO:9: Sorghum bicolor (version XP_002464558.1 GI:242034327)
SEQ NO:11: Zea mays (version NP_001105816.1 GI:162461219)
SEQ NO:13: NP_001105269.1 Zea mays (version NP_001105269.1 GI:162458548)
SEQ NO:15: NP_001266355.1 Zea mays (version NP_001266355.1 GI:525343585)
SEQ NO:17: XP_004983000.1 Setaria italic (version XP_004983000.1 GI:514816524
SEQ NO:19: NP_001048976.1 Oryza sativa Japonica Group (version NP_001048976.1 GI:115450751)
SEQ NO:21: XP_004985679.1 Setaria italic (version XP_004985679.1 GI:514822017)
SEQ NO:23: EAY93198.1 Oryza sativa Indica Group (version EAY93198.1 GI:125547376)
SEQ NO:25: NP_001052194.1 Oryza sativa Japonica Group (version NP_001052194.1 GI:115457188
SEQ NO:27: XP_003558115.1 Brachypodium distachyon (version XP_003558115.1 GI:357112638)
SEQ NO:29: XP_002468495.1 Sorghum bicolor(version XP_002468495.1 GI:242042201
SEQ NO:31: XP_004975146.1 Setaria italic (version XP_004975146.1 GI:514800438
SEQ ID NO:32: EOX94467.1 Theobroma cacao (versionEOX94467.1 GI:508702571; corresponding cDNA: CM001879.1)
SEQ ID NO: 33: XP_002531532.1 Ricinus communis (version XP_002531532.1 GI:255581449, corresponding cDNA:XM_002531486.1)
SEQ ID NO: 34: AFU07481.1 Camellia oleifera (version AFU07481.1 GI:407316573, corresponding cDNA: JX403969.1)
SEQ ID NO: 35: AAF74025.1 Nicotiana tabacum (version AAF74025.1 GI:8248034, corresponding cDNA:AF156696.1)
SEQ ID NO: 36: ADL27918.1 Hevea brasiliensis (version ADL27918.1 GI:302353424; corresponding cDNA:HM015901.1)
SEQ ID NO: 37: XP_006354490.1 Solanum tuberosum (version XP_006354490.1 GI:565375975, corresponding cDNA: XM_006354428.1)
SEQ ID NO:38: XP_002879774.1 Arabidopsis lyrata subsp. Lyrata(version XP_002879774.1 GI:297823783, corresponding cDNA: XM_002879728.1).

FIG. 7: Panicle number, straw dry weight and nutrient elements analysis of transgenic plants expressing PT8^{S517} and PT8^{S517A} under the control of its own promoter in a field experiment with low P soil. (a) Panicle number of the control plant (PT8^{S517}) and the PT8^{S517A} plants. (b) Straw dry weight of the two transgenic plants. (c, and d) Elemental analysis for shoots of the two transgenic plants. The shoots were harvested, washed with deionized water for three times and oven-dried for 3 days at 105° C. for the elements analysis using an inductively coupled plasma optical emission spectrometer (ICP-OES, Optima 8000DV, Perkin-Elmer, USA). No significant differences in the elements were found, with the exception of P and Zn. K, potassium; Ca, calcium; Mg, magnesium; S, sulfate; Fe, iron; Zn, zinc and Mn, manganese. Error bar=s.d. n=3. Data significantly different from the corresponding wild type controls are indicated (** P<0.01; Student's t test). The experiment was conducted in a low P soil field experiment with application of P-fertilizers at the Agricultural Experiment Station of Zhejiang University in Changxin County, Zhejiang (from May to October. 2013). Nitrogen and potassium were applied at usual levels (450 kg urea/ha; 300 kg KCl/ha). The plants were transplanted as 4×5 plants with 25 cm×25 cm with three replications randomly arranged. Fifty plants from each replication were harvested for yield, panicle number and dried straw weight calculation. The soil Olsen P: 7.6 ppm and pH: 6.87 (soil:water=1:1).

### SUMMARY

In a first aspect, the invention relates to a transgenic monocot plant expressing a nucleic acid construct comprising a nucleic acid sequence encoding a mutant PT polypeptide comprising an amino acid modification at position S517 as set forth in SEQ ID No. 2 or of a serine at corresponding position in a sequence that is a functional variant of or homologous to SEQ ID NO. 2.

In another aspect, the invention relates to an isolated nucleic acid encoding a mutant plant PT polypeptide comprising an amino acid substitution at position S517 as set forth in SEQ ID No. 2 or of a serine at an equivalent position in a sequence that is a functional variant of or homologous to SEQ ID NO. 2 wherein said plant is a monocot plant.

In another aspect, the invention relates to a vector comprising an isolated nucleic acid encoding a mutant plant PT polypeptide comprising an amino acid substitution at position S517 as set forth in SEQ ID No. 2 or of a serine at an equivalent position in a sequence that is a functional variant of or homologous to SEQ ID NO. 2 wherein said plant is a monocot plant.

In another aspect, the invention relates to a host cell comprising a nucleic acid a vector according described above.

In another aspect, the invention relates to a method for increasing yield in a transgenic plant comprising introducing and expressing a nucleic acid a vector described above into a plant.

In another aspect, the invention relates to method for increasing Pi use efficiency in a transgenic plant comprising introducing and expressing a nucleic acid a vector described above into a plant.

In another aspect, the invention relates to a method for increasing zinc content in a transgenic plant comprising introducing and expressing a nucleic acid a vector described above into a plant.

In another aspect, the invention relates to a method for producing a transgenic monocot plant with increased yield comprising introducing and expressing a nucleic acid or a vector described above into a plant.

In another aspect, the invention relates to a monocot plant obtained or obtainable by a method described above.

In another aspect, the invention relates to the use of a nucleic acid described above or a described above for increasing yield.

In another aspect, the invention relates to a method for producing a plant with increased yield or increased zinc content comprising the steps of
a) exposing a population of plants to a mutagen and,
b) identifying mutant plants in which the serine at position 517 with reference to SEQ ID No. 2 or a serine at an equivalent position in a sequence homologous to SEQ ID No. 2 is replaced by a to a non-phosphorylatable residue.

In another aspect, the invention relates to a plant obtained or obtainable by a method described above wherein said plant is not Arabidopsis.

In another aspect, the invention relates to a mutant monocot plant having a mutation in a PT gene wherein said mutant PT gene encodes a mutant PT polypeptide comprising an amino acid modification at position S517 as set forth in SEQ ID No. 2 or of a serine at corresponding position in a sequence that is a functional variant of or homologous to SEQ ID NO. 2 generated by generated by mutagenesis.

### DETAILED DESCRIPTION

The present invention provides plants that have increased Pi uptake which does not result in the Pi toxicity syndrome, but surprisingly results in increased yield. The plants are mutant plants that express a PT gene encoding a mutant PT polypeptide with a point mutation in a conserved phosphorylation site. As shown herein, these plants have increased Pi uptake even under low Pi conditions. At the same time and surprisingly, under these conditions, Pi uptake is not increased when wild type (wt) PT is overexpressed. Increased expression of the wt protein does not lead to increased Pi uptake and increased yield under low Pi conditions although such overexpression increases the quantity of the PT protein. Only overexpression of a non-phosphorylatable mutant of PT with a mutation at one of the conserved phosphorylation sites corresponding to a serine (S) residue at 517 in OsPT8 leads to increased Pi uptake. Modifications at other phosphorylation sites do not result in increased Pi uptake and increased yield.

Importantly, the inventors have shown that phosphorylation of a serine residue at position 517 in the OsPT8 peptide does not only affect transit of PT from the ER to the plasma membrane, but notably it also increases stability of PT in the plasma membrane. The non-phosphorylatable mutant PT exits the ER and is more stable in the plasma membrane. Whilst phosphorylation of S514 in AtPHT1:1 has been suggested to impair the recognition of the ER export motif in Arabidopsis, it has also been shown that phosphorylation of S514 in AtPHT1:1 does not affect the degradation of the protein in the PM and does thus not have an effect on stability of the membrane protein. Moreover, it has also been shown that there are differences in the regulation of Pi uptake in the monocot plant rice and in the dicot plant Arabidopsis and overexpression of PHF1 results in an increase of Pi accumulation at high Pi concentration in transgenic rice, but not in Arabidopsis.

The surprising phenotype of the non-phosphorylatable mutant of OsPT8 which leads to increased yield at low Pi conditions can be attributed to the combined increase in exit of the protein from the ER and increase in stability of the protein in the PM. The single modification at one of the conserved phosphorylation sites therefore results in the combined increase in exit of the protein from the ER and increase in stability of the protein in the membrane. It is this combined increase which unexpectedly results in increased Pi uptake and increased yield even under low Pi conditions.

The inventors have also shown that paints expressing a mutant Os PT8 with a mutation at a serine (S) residue at 517 have increased zinc level compared to a control plant (see FIG. 7).

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Preferably, the sequence is cDNA for example as shown in SEQ ID NO: 3.

The terms “peptide”, “polypeptide” and “protein” are used interchangeably herein and refer to amino acids in a polymeric form of any length, linked together by peptide bonds.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or
(b) genetic control sequence(s) which is operably linked with the nucleic acid sequence according to the invention, for example a promoter, or
(c) a) and b)
are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 both incorporated by reference.

A transgenic plant for the purposes of the various aspects of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. According to the invention, the transgene is integrated into the plant in a stable manner and preferably the plant is homozygous for the transgene.

The aspects of the invention pertaining to transgenic plants involve recombination DNA technology and exclude embodiments that are solely based on generating plants by traditional breeding methods.

Other aspects of the invention involve the treatment of plants with a mutagen to produce mutant plants that have appoint mutation in a conserved phosphorylation site. These plants do not carry a PT transgene. However, such methods for producing mutant plants require the step of treating the plants with a mutagen and thus also exclude embodiments that are solely based on generating plants by traditional breeding methods.

The inventors have generated transgenic rice plants which express a mutant OsPT8 polypeptide and which have increased yield and Pi transport. Therefore, these plants use Pi more efficiently than a wt plant and require less fertiliser when used in agriculture than non-modified plants.

The term “yield” includes one or more of the following non-limitative list of features: early flowering time, biomass (vegetative biomass (root and/or shoot biomass) or seed/grain biomass), seed/grain yield, seed/grain viability and germination efficiency, seed/grain size, starch content of grain, early vigour, greenness index, increased growth rate, delayed senescence of green tissue. The term “yield” in general means a measurable produce of economic value, typically related to a specified crop, to an area, and to a period of time. Individual plant parts directly contribute to yield based on their number, size and/or weight. The actual yield is the yield per square meter for a crop and year, which is determined by dividing total production (includes both harvested and appraised production) by planted square metres.

Thus, according to the invention, yield comprises one or more of and can be measured by assessing one or more of: increased seed yield per plant, increased seed filling rate, increased number of filled seeds, increased harvest index, increased viability/germination efficiency, increased number or size of seeds/capsules/pods/grain, increased growth or increased branching, for example in florescences with more branches, increased biomass or grain fill. Preferably, increased yield comprises an increased number of grain/seed/capsules/pods, increased biomass, increased growth, increased number of floral organs and/or floral increased branching. Yield is increased relative to a control plant.

Control plants as defined herein are plants that do not express the nucleic acid or construct described herein, for example wild type plants. The control plant is typically of the same plant species, preferably having the same genetic background as the modified plant.

The terms “increase”, “improve” or “enhance” as used herein are interchangeable. Yield for example is increased by at least a 2%, 3%, 4%, 5%, 6%, 7%, 8%, 9% or 10%, preferably at least 10% to 15%, 15% or 20%, more preferably 25%, 30%, 35%, 40% or 50% or more in comparison to a control plant. For example, yield may be increased by 2% to 50%, for example 10% to 40%.

In a first aspect, the invention relates to a transgenic plant expressing a nucleic acid construct comprising a nucleic acid sequence encoding a mutant PT polypeptide comprising an amino acid modification at position S517 as set forth in SEQ ID No. 2 or of a serine at an equivalent position in a polypeptide sequence that is a functional variant of or homologous to SEQ ID NO. 2 wherein said plant is not Arabidopsis.

Preferably, the invention relates to a transgenic monocot plant expressing a nucleic acid construct comprising a nucleic acid sequence encoding a mutant PT polypeptide comprising an amino acid modification at position S517 as set forth in SEQ ID No. 2 or of a serine at an equivalent position in a polypeptide sequence that is a functional variant of or homologous to SEQ ID NO. 2.

The invention also relates to a method for increasing yield or zinc content/level in a transgenic plant comprising introducing and expressing a nucleic acid construct comprising a nucleic acid encoding a mutant plant PT polypeptide comprising an amino acid modification at position S517 as set forth in SEQ ID No. 2 or of a serine at an equivalent position in a sequence that is a functional variant of or homologous to SEQ ID NO. 2. In one embodiment, said plant is not Arabidopsis.

Zinc content/level can be increased at least 2 fold compared to a wild type plant.

The invention also relates to a method for increasing yield in a transgenic monocot plant comprising introducing and expressing a nucleic acid construct comprising a nucleic acid encoding a mutant plant PT polypeptide comprising an amino acid modification at position S517 as set forth in SEQ ID No. 2 or of a serine at an equivalent position in a sequence that is a functional variant of or homologous to SEQ ID NO. 2.

The invention also relates to a method for increasing Pi uptake in a transgenic plant comprising introducing and expressing a nucleic acid construct comprising a nucleic acid encoding a mutant plant PT polypeptide comprising an amino acid modification at position S517 as set forth in SEQ ID No. 2 or of a serine at an equivalent position in a sequence that is a functional variant of or homologous to SEQ ID NO. 2. In one embodiment, said plant is not Arabidopsis.

The invention also relates to a method for increasing Pi uptake in a transgenic monocot plant comprising introducing and expressing a nucleic acid construct comprising a nucleic acid encoding a mutant plant PT polypeptide comprising an amino acid substitution at position S517 as set forth in SEQ ID No. 2 or of a serine at an equivalent position in a sequence that is a functional variant of or homologous to SEQ ID NO. 2.

The invention also relates to a method alleviating zinc deficiency in a transgenic plant, preferably a monocot plant, comprising introducing and expressing a nucleic acid construct comprising a nucleic acid encoding a mutant plant PT polypeptide comprising an amino acid substitution at position S517 as set forth in SEQ ID No. 2 or of a serine at an equivalent position in a sequence that is a functional variant of or homologous to SEQ ID NO. 2.

The modification/mutation in the PT mutant polypeptides according to the various aspects of the invention described herein is with reference to the amino acid position as shown in SEQ NO. 2 which designates the OsPT8 wild type polypeptide sequence. In the wt OsPT8 sequence, the target serine residue is located at position 517. The wt polypeptide is encoded by the wild type (wt) nucleic acid shown in SEQ ID No. 1 or SEQ ID No. 3 (cDNA sequence) respectively. Thus, in one embodiment according to the various aspects of the invention, the mutant PT polypeptide is encoded by a nucleic acid comprising or consisting of a sequence substantially identical to SEQ ID No. 1, a functional variant, ortholog or homolog thereof, but which has a modification of a codon so that transcription of the nucleic acid results in a mutant protein comprising an amino acid modification corresponding to position S517 as set forth in SEQ ID No. 2 or corresponding to a serine at an equivalent position. In other words, the mutant PT polypeptide is encoded by a nucleic acid comprising or consisting of a sequence substantially identical to SEQ ID No. 1 or 3, a functional variant, ortholog or homolog thereof, but comprises a modification in the codon encoding S517 as set forth in SEQ ID No. 2 or a serine at an equivalent position.

The modification at position 517 in OsPT8 or at of a serine at an equivalent position in a homolog can be a deletion of the serine residue. Preferably, the modification is a substitution of serine with another amino acid residue that is non-phosphorylatable. For example, this residue is alanine (A) or any other suitable amino acid.

In one embodiment of the various aspects of the invention, the PT mutant polypeptide is a mutant PT polypeptide of OsPT8 as shown in SEQ ID No. 2 but comprising an amino acid substitution at position S517 in SEQ ID No. 2. Accordingly, the nucleic acid encoding said peptide is substantially identical to OsPT8 as shown in SEQ ID No. 1, and encodes a mutant polypeptide but comprising an amino acid modification if serine at position 517 of SEQ ID No. 2. In one embodiment, the modification is a substitution. The S residue at position 517 may be substituted with A or any other suitable amino acid.

However, the various aspects of the invention also extend to homologs and variants of OsPT8. As used herein, these are functional homologs and variants. A functional variant or homolog of OsPT8 as shown in SEQ ID No. 2 is a PT polypeptide which is biologically active in the same way as SEQ ID No. 2, in other words, it is a Pi transporter and regulates Pi uptake. The term functional homolog or homolog as used herein includes OsPT8 orthologs in other plant species. In a preferred embodiment of the various aspects of the invention, the invention relates specifically to OsPT8 or orthologs of OsPT8 in other plants. Orthologs of OsPT8 in monocot plants are preferred. A variant has a modified sequence compared to the wild type sequence, but this does not affect the functional activity of the protein. A skilled person would know that amino acid substitutions in parts of the protein that do not include functional motifs are less likely to affect protein function. Preferably, a variant as used herein has at least 90%, 91%, 92%, 93%, 94%, 95%, 96%, 97%, 98%, or 99% overall sequence identity to the wild amino acid or nucleic acid sequence.

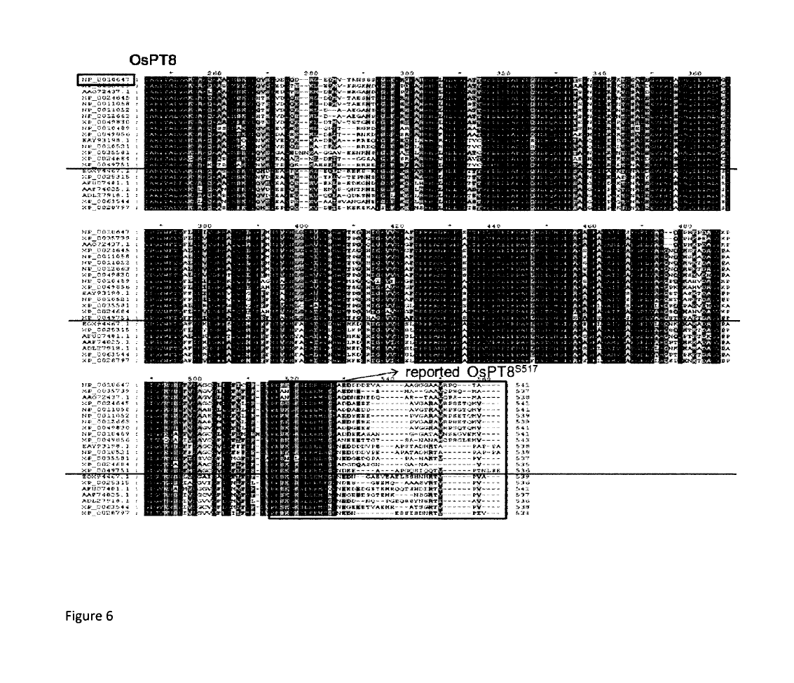

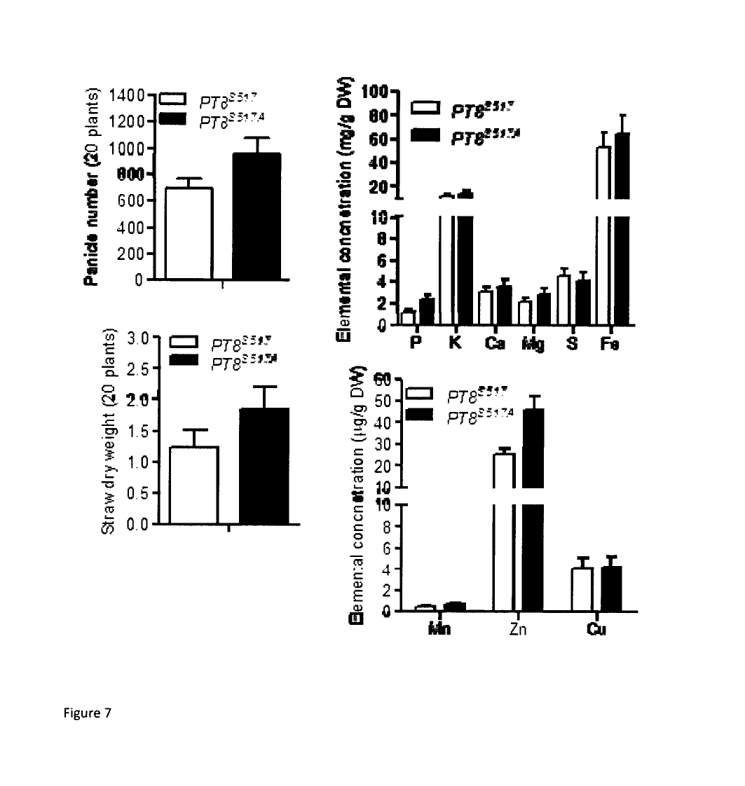

As explained below, other PT polypeptides share sequence homology with OsPT8 and residues for manipulation that correspond to position S517 in OsPT8 can be readily identified in these homologs by sequence comparison and alignment. This is illustrated in FIG. 6 which identifies sequences of homologous PT polypeptides in monocot plants and highlights the conserved phosphorylation site at S517 in OsPT8 and the equivalent/corresponding serine residue in homologous sequences.

According to the various aspects of the invention, the homolog of a OsPT8 polypeptide has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2. Preferably, overall sequence identity is 90%, 91%, 92%, 93%, 94%, 95%, 96%, 97%, 98%, or 99%. In another embodiment, the homolog of a OsPT8 nucleic acid sequence has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid represented by SEQ ID NO: 1 or 3. Preferably, overall sequence identity is 90%, 91%, 92%, 93%, 94%, 95%, 96%, 97%, 98%, or 99%. The overall sequence identity is determined using a global alignment algorithm known in the art, such as the Needleman Wunsch algorithm in the program GAP (GCG Wisconsin Package, Accelrys). Non-limiting examples of such amino acid sequences are shown in FIG. 6. Thus, an otholog may be selected from SEQ ID NO. 5, 7, 9, 11, 13, 15 1, 17, 19, 21, 23, 25, 27, 29, 31, 32, 33, 34, 35, 36, 37, 38 as shown in FIG. 6 or SEQ No. 40 from wheat. Nucleic acids for monoct species that can be used transformation and which have the mutation at the corresponding serine position are shown in SEQ ID NOs: 4, 6, 8, 10, 12, 14, 16, 18, 20, 22, 24, 26, 28, and 30 or SEQ No. 39 from wheat. Also included are functional variants of these homolog sequences which have at least 90%, 91%, 92%, 93%, 94%, 95%, 96%, 97%, 98% or 99% overall sequence identity to the homologous amino acid sequences.

Preferably, the OsPT8 homolog has the following conserved motifs, for example an “EXE”-ER exit motif as well as the motif “SLEE” (512-515aa of OsPT8, a casein kinase II target site) and the serine 517 in OsPT8 adjacent to “SLE”.

Suitable homologs can be identified by sequence comparisons and identifications of conserved domains. The function of the homolog can be identified as described herein and a skilled person would thus be able to confirm the function when expressed in a plant. Thus, one of skill in the art will recognize that analogous amino acid substitutions listed above with reference to SEQ ID No. 2 can be made in PT from other plants by aligning the OsPT8 polypeptide sequence to be mutated with the OsPT8 polypeptide sequence as set forth in SEQ ID NO: 2.

As a non-limiting example, an amino acid substitution in PT that is analogous to/corresponds to or is equivalent to the amino acid substitution S517 in OsPT8 as set forth in SEQ ID NO: 2 can be determined by aligning the amino acid sequences of OsPT8 (SEQ ID NO:2) and a PT amino acid sequence from another plant species and identifying the position corresponding to S517 in the OsPT8 from another monocot plant species as aligning with amino acid position S517 of OsPT8. This is shown in FIG. 6.

For example, according to the various aspects of the invention, a nucleic acid encoding a mutant PT which is a mutant version of the endogenous PT peptide in a plant may be expressed in said plant by recombinant methods. For example, in one embodiment of the transgenic plants of the invention, the transgenic plant is a rice plant expressing a nucleic acid construct comprising a nucleic acid sequence encoding a mutant PT polypeptide as shown in SEQ ID NO. 2 but comprising an amino acid substitution of S at position S517 with a non-phosphorylatable residue. In another example, the transgenic plant is a transgenic wheat plant expressing a nucleic acid construct comprising a nucleic acid sequence encoding a mutant wheat OsPT8 homolog polypeptide as shown in SEQ ID NO. 2 but comprising an amino acid substitution of a serine residue at a position equivalent to S517 in OsPT8 with a non-phosphorylatable residue. In another example, the transgenic is a maize plant expressing a nucleic acid construct comprising a nucleic acid sequence encoding a mutant maize OsPT8 homolog polypeptide as shown in SEQ ID NO. 2 but comprising an amino acid substitution of a serine residue at a position equivalent to S517 in OsPT8 with a non-phosphorylatable residue. In another example, the transgenic is a barley plant expressing a nucleic acid construct comprising a nucleic acid sequence encoding a mutant barley OsPT8 homolog polypeptide as shown in SEQ ID NO. 2 but comprising an amino acid substitution of a serine residue at a position equivalent to S517 in OsPT8 with a non-phosphorylatable residue.

In another embodiment, a mutant PT which is a mutant version of a PT peptide in one plant may be expressed exogenously in a second species as defined herein by recombinant methods. Preferably, the PT is a monocot PT and the plant in which it is expressed is also a monocot plant. For example, OsPT8 may be expressed in another monocot crop plant.

According to the various aspects of the invention, a monocot plant is, for example, selected from the families Arecaceae, Amaryllidaceae, Graminseae or Poaceae. For example, the plant may be a cereal crop. A cereal crop may be selected from wheat, rice, barley, maize, oat, sorghum, rye, millet, buckwheat, turf grass, Italian rye grass, sugarcane, or Festuca species, or a crop such as onion, leek, yam, pineapple or banana. This list is non-limiting and other monocot plants are also within the scope of the various aspects and embodiments of the invention.

In one embodiment of the various aspects of the invention, the PT polypeptide may comprise additional modifications. In another embodiment, the polypeptide does not comprise further modifications.

In one embodiment of the transgenic plant of the invention, the plant may express additional transgenes.

According to the various aspects of the invention, including the methods, plants and uses described herein, the nucleic acid construct expressed in the transgenic plant may comprise a regulatory sequence. The terms “regulatory element”, “regulatory sequence”, “control sequence” and are all used interchangeably herein and are to be taken in a broad context to refer to regulatory nucleic acid sequences capable of effecting expression of the sequences to which they are ligated. Such sequences are well known in the art.

The regulatory sequence can be a promoter. The term “promoter” typically refers to a nucleic acid control sequence located upstream from the transcriptional start of a gene and which is involved in recognising and binding of RNA polymerase and other proteins, thereby directing transcription of an operably linked nucleic acid. The term “regulatory element” also encompasses a synthetic fusion molecule or derivative that confers, activates or enhances expression of a nucleic acid molecule in a cell, tissue or organ. Furthermore, the term “regulatory element” includes downstream transcription terminator sequences. A transcription terminator is a section of nucleic acid sequence that marks the end of a gene or operon in genomic DNA during transcription. Transcription terminator used in construct to express plant genes are well known in the art.

In one embodiment, the constructs described herein have a promoter and a terminator sequence.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence described herein. This also applies to other “plant” regulatory signals, such as “plant” terminators.

The promoters upstream of the PT nucleotide sequences useful in the aspects of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule is, as described above, advantageously linked operably to or comprises a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest.

Many promoters used to express plant genes in plants are known in the art. The below is a non-limiting list and a skilled person would be able to choose further embodiments form those known in the art.

A “constitutive promoter” refers to a promoter that is transcriptionally active during most, but not necessarily all, phases of growth and development and under most environmental conditions, in at least one cell, tissue or organ. Examples of constitutive promoters include but are not limited to actin, HMGP, CaMV19S, GOS2, rice cyclophilin, maize H3 histone, alfalfa H3 histone, 34S FMV, rubisco small subunit, OCS, SAD1, SAD2, nos, V-ATPase, super promoter, G-box proteins and synthetic promoters.

A “strong promoter” refers to a promoter that leads to increased or overexpression of the gene. Examples of strong promoters include, but are not limited to, CaMV-35S, CaMV-35S omega, Arabidopsis ubiquitin UBQ1, rice ubiquitin, actin, or Maize alcohol dehydrogenase 1 promoter (Adh-1). The term “increased expression” or “overexpression” as used herein means any form of expression that is additional to the control, for example wild-type, expression level. In one embodiment of the various aspects of the invention, the promoter is CaMV-35S.

In another embodiment, the regulatory sequence is an inducible promoter, a stress inducible promoter or a tissue specific promoter. The stress inducible promoter is selected from the following non limiting list: the HaHB1 promoter, RD29A (which drives drought inducible expression of DREB1A), the maize rabI7 drought-inducible promoter, P5CS1 (which drives drought inducible expression of the proline biosynthetic enzyme P5CS1), ABA- and drought-inducible promoters of Arabidopsis clade A PP2Cs (ABI1, ABI2, HAB1, PP2CA, HAI1, HAI2 and HAI3) or their corresponding crop orthologs.

The promoter may also be tissue-specific.

In a one embodiment, the promoter is a constitutive or strong promoter, such as CaMV-35S.

As mentioned above, the invention also relates to methods for increasing yield by expressing a mutant PT nucleic acid as described herein. The invention thus relates to a method for increasing yield in a transgenic plant comprising introducing and expressing a nucleic acid construct comprising a nucleic acid encoding a mutant plant PT polypeptide comprising an amino acid modification at position S517 as set forth in SEQ ID No. 2 or of a serine at an equivalent position in a sequence that is a functional variant of or homologous to SEQ ID NO. 2 wherein said plant is not Arabidopsis. Thus, the plant may be a dicot plant, but not Arabidopsis.

The invention also relates to a method for increasing yield in a transgenic monocot plant comprising introducing and expressing a nucleic acid construct comprising a nucleic acid encoding a mutant plant PT polypeptide comprising an amino acid modification at position S517 as set forth in SEQ ID No. 2 or of a serine at an equivalent position in a sequence that is a functional variant of or homologous to SEQ ID NO. 2. In one embodiment, the nucleic acid encodes a polypeptide as shown in SEQ ID NO. 2 but wherein serine at position 517 in SEQ ID No. 2 is substituted. In another embodiment, the nucleic acid encodes a polypeptide that is homolog of SEQ ID NO. 2 and comprises a substitution of a serine at a position equivalent to S517 in SEQ ID No. 2. In one embodiment, the nucleic acid encodes a polypeptide as shown in SEQ ID NO. 2 but wherein serine at position 517 in SEQ ID No. 2 is substituted and the plant is rice.

The invention also relates to a method for increasing Pi uptake in a transgenic plant comprising introducing and expressing a nucleic acid construct comprising a nucleic acid encoding a mutant plant PT polypeptide comprising an amino acid modification corresponding to position S517 as set forth in SEQ ID No. 2 or corresponding to an equivalent position in a sequence that is a functional variant of or homologous to SEQ ID NO. 2 wherein said plant is not Arabidopsis. Thus, the plant may be a dicot plant, but not Arabidopsis.

The invention also relates to a method for increasing Pi uptake in a transgenic monocot plant comprising introducing and expressing a nucleic acid construct comprising a nucleic acid encoding a mutant plant PT polypeptide comprising an amino acid modification corresponding to position S517 as set forth in SEQ ID No. 2 or corresponding to an equivalent position in a sequence that is a functional variant of or homologous to SEQ ID NO. 2. In one embodiment, the nucleic acid encodes a polypeptide as shown in SEQ ID NO. 2 but wherein serine at position 517 in SEQ ID No. 2 is substituted. In another embodiment, the nucleic acid encodes a polypeptide that is homolog of SEQ ID NO. 2 and comprises a substitution of a serine at a position equivalent to S517 in SEQ ID No. 2. In one embodiment, the nucleic acid encodes a polypeptide as shown in SEQ ID NO. 2 but wherein serine at position 517 in SEQ ID No. 2 is substituted and the plant is rice.

The invention also relates to a method for increasing Pi use efficiency in a transgenic plant comprising introducing and expressing a nucleic acid construct comprising a nucleic acid encoding a mutant plant PT polypeptide comprising an amino acid modification corresponding to position S517 as set forth in SEQ ID No. 2 or corresponding to an equivalent position in a sequence that is a functional variant of or homologous to SEQ ID NO. 2 wherein said plant is not Arabidopsis. Thus, the plant may be a dicot plant, but not Arabidopsis.

The invention also relates to a method for increasing Pi use efficiency in a transgenic monocot plant comprising introducing and expressing a nucleic acid construct comprising a nucleic acid encoding a mutant plant PT polypeptide comprising an amino acid modification corresponding to position S517 as set forth in SEQ ID No. 2 or corresponding to an equivalent position in a sequence that is a functional variant of or homologous to SEQ ID NO. 2. In one embodiment, the nucleic acid encodes a polypeptide as shown in SEQ ID NO. 2 but wherein serine at position 517 in SEQ ID No. 2 is substituted. In another embodiment, the nucleic acid encodes a polypeptide that is homolog of SEQ ID NO. 2 and comprises a substitution of a serine at a position equivalent to S517 in SEQ ID No. 2. In one embodiment, the nucleic acid encodes a polypeptide as shown in SEQ ID NO. 2 but wherein serine at position 517 in SEQ ID No. 2 is substituted and the plant is rice.

Preferably, the modification of the serine residue in the method above is a substitution with a non-phosphorylatable residue, such as A.

In one embodiment of the methods described above, the nucleic acid construct comprises one or more regulatory sequence as described herein. This can be a 35S promoter.

As described above, according to these methods, a modified endogenous nucleic acid encoding a mutant PT polypeptide which is a mutant version of the endogenous PT polypeptide in a plant may be expressed in said plant by recombinant methods. For example, in one embodiment the method comprises expressing a nucleic acid construct comprising a nucleic acid sequence encoding a mutant PT polypeptide as shown in SEQ ID NO. 2 but comprising an amino acid substitution at position S517 in rice. In another example, the method comprises expressing a nucleic acid construct comprising a nucleic acid sequence encoding a mutant wheat OsPT8 homolog polypeptide comprising an amino acid substitution of a serine residue at a position equivalent to S517 in OsPT8 in wheat. In another example, the method comprises expressing a nucleic acid construct comprising a nucleic acid sequence encoding a mutant maize OsPT8 homolog polypeptide comprising an amino acid substitution of a serine residue at a position equivalent to S517 in OsPT8 in maize. In another example, the method comprises expressing a nucleic acid construct comprising a nucleic acid sequence encoding a mutant barley OsPT8 homolog polypeptide comprising an amino acid substitution of a serine residue at a position equivalent to S517 in OsPT8 in barley.

In another embodiment, a mutant PT which is a mutant version of a PT peptide in one plant may be expressed exogenously in a second plant of another species as defined herein by recombinant methods. Preferably, the PT is a monocot PT and the plant in which it is expressed is also a monocot plant. For example, OsPT8 may be expressed in another monocot crop plant.

The methods of the invention described above may also optionally comprise the steps of screening and selecting plants for those that comprise a polynucleotide construct as above compared to a control plant. Preferably, according to the methods described herein, the progeny plant is stably transformed and comprises the transgenic polynucleotide which is heritable as a fragment of DNA maintained in the plant cell and the method may include steps to verify that the construct is stably integrated. The method may also comprise the additional step of collecting seeds from the selected progeny plant. A further step can include assessing and/or measuring yield and/or Pi uptake.

In one embodiment, yield and Pi uptake are increased under low Pi conditions in the soil.

Phosphorous is one of the least available essential nutrients in the soil. Plants can only assimilate inorganic Pi. Available Pi in the soil is influenced by various factors, in particular soil pH which determines the solubility of Pi, but also minerals such as silica, iron and aluminium, all of which tightly bind Pi. Other factors such as the level of phytic acid, for example as found in poultry manure and derived from plant material in fed), since phytate binds phosphate and as such is unavailable for uptake by the roots. Free Pi levels in soil ranges from 2 uM or less up to 10 uM in fertile soils. Soil Pi levels of less than 10 uM are generally considered to be low Pi. These levels are much lower than the levels of Pi in plant tissues. Pi levels varying between plant cellular compartments—typically 80-80 um in the cytoplasm, and 2-8 mM in organelles and as much as 35-75 mM in the vacuole (see Raghothama).

Large areas of global agriculture, such as those of eastern USA, SE Asia, central and eastern Europe, central Africa and others have soil acidity and other factors that acutely bind Pi. FAO data for fertilizer consumption indicate widely different practices in global agriculture, ranging from as little as 2 kg per hectare in Angola or Uganda, through 46 kg/Ha (Australia), 120 Kg/Ha (USA), 217 Kg/Ha (Pakistan), 251 Kg/Ha (UK) to 1,272 Kh/Ha (New Zealand)

In defining the levels of Pi, even in soils with higher Pi levels, the level of annually applied Pi fertilizer is taken into account. For example, application of only 50-60% of the levels of Pi fertilizer normally applied by farmers in a particular region/crop would be regarded as low Pi situation for crop growth.

Thus, as used herein, low Pi conditions for crop growth can be defined as Pi levels of less than 10 uM. Low Pi conditions can also be defined as situations where 50-60% of the levels of Pi fertilizer normally applied by farmers in a particular region/crop.

The invention also relates to an isolated mutant nucleic acid encoding a mutant plant PT polypeptide comprising an amino acid modification of serine position S517 as set forth in SEQ ID No. 2 or of a serine at an equivalent position in a sequence that is a functional variant of or homologous to SEQ ID NO. 2 wherein said plant is a monocot plant. Homologs of SEQ ID No. 2 are defined elsewhere herein.

The modification is preferably a substitution of the serine residue with a non-phosphorylatable residue which renders the polypeptide non-phosphorylatable at that location.

In one embodiment, the isolated mutant nucleic acid is cDNA. For example, the isolated mutant nucleic acid is cDNA corresponds to SEQ ID No. 3, but has a mutation at the codon coding for S517. In another embodiment, the isolated mutant nucleic acid is cDNA corresponds to SEQ ID No. 4, 6, 8, 10, 12, 14, 16, 18, 20, 22, 24, 26, 28, 30 or 39, but has a mutation at the codon coding for an amino acid at an equivalent position to S517 in SEQ ID No. 2.

In one embodiment, the isolated mutant nucleic acid encodes a polypeptide substantially as shown in SEQ ID NO. 2 but wherein serine at position 517 in SEQ ID No. 2 is substituted. The isolated wild type nucleic acid is shown in SEQ ID No. 1, but the mutant nucleic acid which forms part of the invention includes a substitution of one or more nucleic acid in the codon encoding serine 571 in OsPT8 or in an equivalent codon.

The invention also extends to a vector comprising an isolated mutant nucleic acid described above. The vector may comprise one or more regulatory sequence which directs expression of the nucleic acid. The term regulatory sequence is defined elsewhere herein. In one embodiment, a regulatory sequence is the 35S promoter.

The invention also relates to an isolated host cell transformed with a mutant nucleic acid or vector as described above. The host cell may be a bacterial cell, such as Agrobacterium tumefaciens, or an isolated plant cell wherein said plant is not Arabidopsis and preferably is a monocot plant cell as defined herein. In one embodiment, the plant cell is a rice cell which expresses an isolated mutant nucleic acid encodes a polypeptide substantially as shown in SEQ ID NO. 2 but wherein serine at position 517 in SEQ ID No. 2 is substituted.

The invention also relates to a culture medium or kit comprising a culture medium and an isolated host cell as described above.

The invention also relates to the use of a nucleic acid or vector described above for increasing yield of a plant, preferably of a monocot plant. In one embodiment, the nucleic acid encodes a polypeptide as shown in SEQ ID NO. 2 but wherein serine at position 517 in SEQ ID No. 2 is substituted with another amino acid. In one embodiment, the nucleic acid encodes a polypeptide as shown in SEQ ID NO. 2 but wherein serine at position 517 in SEQ ID No. 2 is substituted and the plant is rice. In another embodiment, the nucleic acid is a homolog of SEQ ID NO. 2, preferably form a monocot plant, but wherein serine at a position equivalent to 517 in SEQ ID No. 2 is substituted with another non-phosphorylatable amino acid.

The nucleic acid or vector described above is used to generate transgenic plants, specifically the transgenic plants described herein, using transformation methods known in the art. Thus, according to the various aspects of the invention, a nucleic acid comprising a sequence encoding for a mutant PT polypeptide as described herein, is introduced into a plant and expressed as a transgene. The nucleic acid sequence is introduced into said plant through a process called transformation. The term “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, mega gametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and micro projection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker. Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The invention also relates to a method for producing a transgenic monocot plant with increased yield comprising introducing and expressing a nucleic acid or vector described above into a plant wherein said plant is not Arabidopsis. Preferably, said plant is a monocot plant as defined elsewhere herein. In one embodiment, the nucleic acid encodes a polypeptide as shown in SEQ ID NO. 2 but wherein serine at position 517 in SEQ ID No. 2 is substituted with another amino acid. In one embodiment, the nucleic acid encodes a polypeptide as shown in SEQ ID NO. 2 but wherein serine at position 517 in SEQ ID No. 2 is substituted and the plant is rice. In another embodiment, the nucleic acid is a homolog of SEQ ID NO. 2 but wherein serine at a position equivalent to 517 in SEQ ID No. 2 is substituted with another amino acid.

The term “plant” as used herein encompasses whole plants, ancestors and progeny of the plants and plant parts, including seeds/grain, fruit, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

The various aspects of the invention described herein clearly extend to any plant cell or any plant produced, obtained or obtainable by any of the methods described herein, and to all plant parts and propagules thereof unless otherwise specified. For example, in certain aspects described above, rice is specifically excluded. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also extends to harvestable parts of a plant of the invention as described above such as, but not limited to seeds/grain, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, flour, starch or proteins. The invention also relates to food products and food supplements comprising the plant of the invention or parts thereof.

Arabidopsis is specifically disclaimed from some of the aspects of the invention. Thus, the transgenic plants of the invention do not encompass Arabidopsis. In other embodiments, dicot plants are specifically disclaimed from some of the aspects of the invention. For example, in one embodiment of the transgenic plants of the invention, these exclude dicots. As also described above, the preferred aspects of the invention, including the transgenic plants, methods and uses, relate to monocot plants.

In other aspects of the invention, plants having increased yield due to a point mutation at S517 with reference to SEQ ID 2 or at a serine at an equivalent position in a sequence homologous to SEQ ID No. 2 may be produced by random mutagenesis. In these plants, the endogenous PT target gene is mutated and S at position 517 with reference to SEQ ID 2 or a serine at an equivalent position in a sequence homologous to SEQ ID No. 2 is replaced with an amino acid residue that is not phosphorylated. Depending on the method of mutagenesis, the method includes the subsequent steps of screening of mutants to identify mutants with a mutation in the target location and optionally screening for increased yield and increased Pi uptake or screening for increased yield and increased Pi uptake followed by screening of mutants to identify mutants with a mutation in the target location.

Plants that have been identified in the screening steps are isolated and propagated.

Suitable techniques for mutagenesis are well known in the art and include Targeting Induced Local Lesions IN Genomes (TILLING). TILLING is a high-throughput screening technique that results in the systematic identification of non-GMO-derived mutations in specific target genes. Those skilled in the art will also appreciate that TILLING permits the high-throughput identification of mutations in target genes without production of genetically modified organisms and it can be an efficient way to identify mutants in a specific gene that might not confer a strong phenotype by itself), may be carried out to produce plants and offspring thereof with the desired mutation resulting in a change in yield and Pi uptake, thereby permitting identification of non-transgenic plants with advantageous phenotypes.

In one embodiment, the method used to create and analyse mutations is targeting induced local lesions in genomes. In this method, seeds are mutagenised with a chemical mutagen. The mutagen may be fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Another method is CRISP-Cas (19.20).

The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the PT target gene using any method that identifies heteroduplexes between wild-type and mutant genes. For example, denaturing high pressure liquid chromatography (dHPLC), constant denaturant capillary electrophoresis (CDCE), temperature gradient capillary electrophoresis (TGCE), or fragmentation using chemical cleavage can be used.

Preferably, the PCR amplification products are incubated with an endonuclease that preferentially cleaves mismatches in heteroduplexes between wild-type and mutant sequences. Cleavage products are electrophoresed using an automated sequencing gel apparatus, and gel images are analyzed with the aid of a standard commercial image-processing program. Any primer specific to the PT gene may be utilized to amplify the PT genes within the pooled DNA sample. Preferably, the primer is designed to amplify the regions of the PT gene where useful mutations are most likely to arise, specifically in the areas of the PT gene that are highly conserved and/or confer activity. To facilitate detection of PCR products on a gel, the PCR primer may be labelled using any conventional labelling method.

Rapid high-throughput screening procedures thus allow the analysis of amplification products for identifying a mutation conferring increased yield, in particular under low Pi conditions, and increased Pi uptake, as compared to a corresponding non-mutagenised wild-type plant. Once a mutation at S517 with reference to SEQ 2 to a non-phosphorylatable residue, such as A, or at a serine at an equivalent position in a sequence homologous to SEQ ID No. 2 is identified in a PT gene of interest, the seeds of the M2 plant carrying that mutation are grown into adult M3 plants and can optionally be screened for the phenotypic characteristics associated with the PT gene. Mutants with increased yield and increased Pi use efficiency can thus be identified.

A plant produced or identified as described above may be sexually or asexually propagated or grown to produce off-spring or descendants. Off-spring or descendants of the plant regenerated from the one or more cells may be sexually or asexually propagated or grown. The plant or its off-spring or descendants may be crossed with other plants or with itself.

Thus, the invention relates to a method of producing a mutant plant having one or more of increased yield, increased Pi uptake and increased Pi use efficiency comprising: exposing a population of plants to a mutagen and identifying mutant plants in which the serine at position 517 with reference to SEQ ID No. 2 or a serine at an equivalent position in a sequence homologous to SEQ ID No. 2 is replaced by a to a non-phosphorylatable residue.

The method uses the steps of analysing DBA samples from said plant population exposed to a mutagen to identify the mutation as described above. Additional steps may include: determining yield of the mutant plant and comparing said yield to control plants, determining Pi uptake of the mutant plant and comparing said yield to control plants, determining Pi use efficiency of the mutant plant and comparing said yield to control plants. Yield, Pi uptake or Pi use efficiency are preferably assessed under low Pi conditions. Further steps include sexually or asexually propagating a plant produced or identified as described above may be or grown to produce off-spring or descendants.

In a preferred embodiment, the plant is a monocot plant as defined herein, for example rice.

Plants obtained or obtainable by such method which carry a functional mutation in the endogenous PT locus are also within the scope of the invention provided the plant is not Arabidopsis. In a preferred embodiment, the plant is a monocot plant as defined herein, for example rice.

Thus, the invention also relates to a mutant plant having a mutation in a PT gene wherein said mutant PT gene encodes a mutant PT polypeptide comprising an amino acid modification at position S517 as set forth in SEQ ID No. 2 or of a serine at corresponding position in a sequence that is a functional variant of or homologous to SEQ ID NO. 2. The mutant plant is non-transgenic and generated by mutagenesis. The plant is not Arabidopsis. In a preferred embodiment, the plant is a monocot plant as defined herein, for example rice.

The modification is preferably a substitution of the serine residue with a non-phosphorylatable amino acid residue.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents, explicitly including any sequence Id/accession/version numbers mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The invention is further described in the following non-limiting examples.

### EXAMPLES

### Material and Methods

### Plant Materials and Growth Conditions.

Rice cultivars (japonica, Nipponbare: NIP and Xiushui 134: XS134)) as wild-type rice and transgenic plants with knockdown of CK2α3 and CKβ3 were grown hydroponically in a greenhouse with a 12 h day (30° C.)/12 h night (22° C.) photoperiod, approximately 200 μmol m⁻²s⁻¹ photon density, and approximately 60% humidity. Plants with Pi-sufficient and low Pi treatments were prepared by growing them at 200, 50 and 20 μM NaH2PO4, respectively, unless specified otherwise. Tobacco plants (Nicotiana benthamiana) were cultivated ingrowth chambers as described before (21). Field experiment was conducted at low P soil plot at Agricultural Experiment Station of Zhejiang University in Changxing County, Zhejiang province.

Rice Root cDNA Library Construction and Split-Ubiquitin Membrane Yeast Two-Hybrid Screening System.

Total RNA was prepared from roots of 14-d-old seedlings grown in a normal hydroponic solution using the RNeasy Plant Mini kit (Qiagen, Hilden, Germany). Isolated RNA was treated with RNase-free Dnase (Qiagen, Hilden, Germany) and sent to Dualsystems Biotech (Switzerland) for DUAL hunter library construction service. Briefly, 1st strand cDNA generated by reverse transcription was normalized and confirmed by quantitative PCR using two marker genes (OsActin and OsGAPDH). Then, the normalized 1st strand cDNA was size-selected and split into two size pools to optimize representation of big and small fragment. The 2nd strand cDNA was generated separately on both size pools and directionally integrated into prey vector pPR3-N between two variable Sfi I sites.

Ultimately, normalized root of rice cDNA library with 2.9×106 independent clones was obtained. In situations where PT8-protein interactions liberate LexA-VP16 by ubiquitin-specific protease, LexA-VP16 enters the nucleus and interacts with LexA-binding sites, leading to activation of transcription of the ADE2, HIS3 reporter genes. To minimize background arising from nonspecific release of LexA-VP16, which caused histidine selection leakage and activation of the HIS3 reporter gene, we transfected library cDNAs into integrated yeast cell lines mentioned above and made selection on Leucine-Tryptophan-Histidine-Adenine dropout selection plates with 7.5 mM 3-aminotriazole, a competitive inhibitor of the imidazoleglycerolphosphatedehydratase involved in histidine biosynthesis. As a result, we identified multiple independent cDNAs encoding a full-length casein kinase beta subunit protein. In order to verify this hit, pBT3-STE-PT2/8 and positive prey plasmid were transfected back into NMY51. The coexpression of both vectors resulted in yeast growth on selection plates (-Leu-Trp-His-Ade) containing 7.5, or even 10 mM 3-AT but not the negative controls. Thus, the positive clones selected on selection plates containing 7.5 mM 3-aminotriazole were due to the association between PT2/8 and the casein kinase beta subunit. Yeast split-ubiquitination assay. cDNA fragments encoding full length of OsPT2 and OsPT8 (PT2/8), and four CK2 subunits: α2, α3, β1 and β3 were obtained by RT-PCR with the primers PT2-pBT3-STE-U/L and CK2α2/α3/β1/β3-pPR3-N-U/L, respectively, digested by SfiI, and then inserted into pBT3-STE or pPR3-N (DUAL membrane, Schlieren, Switzerland) to generate PT2/8-pBT3-STE, and CK2α2/α3/β1/β3-pPR3-N. The S517A or S517D mutations in full length PT8 were generated with the primers PT8A-P1/2/3/4 and PT8D-P1/2/3/4, while PHF1 was amplified by RT-PCR with primers PHF1-pBT3-N-U/L, then the full length PT8 fragments containing the mutations and wild type PHF1 were cloned into the pPR3-STE and pBT3-N vector to generate PT8S517A/S517D-pPR3-STE and PHF-pBT3-N plasmids, respectively.

### Co-Immunoprecipitation Assays.

cDNA fragments encoding C-terminal (CT) peptides of PT2&PT8 (28/36aa) and the S517A or S517D mutations in PT8-CT were inserted into pCAMBIA1300-GFP vector (22) to generate fusions with GFP. Full length CK2α3/β3 cDNA were inserted into the pF3ZPY122 (23) to generate the CK2α3/β3-pF3ZPY122 plasmids. The CK2β3 coding region and NH2 terminus of PHF1 (coding sequence of hydrophilic WD40 domain of PHF1) were cloned into the pDONR201 plasmid using the Gateway® BP reaction (Life Technologies, Darmstadt, Germany). At this stage, DNA sequence analysis was performed. The transfer of CK2β3 and N-terminus of PHF1 from the pDONR201 plasmid to the pC-TAPα vector (24) was performed using Gateway® LR reaction. The expression vectors were introduced into the Agrobacterium strain EHA105. Individual combinations of plasmids were co-infiltrated into tobacco (Nicotiana benthamiana) leaves as previously described and grown for 3 days. Protein extraction and coimmunoprecipitation were performed as described (25). Immunoprecipitation products were boiled for 5 min and separated by electrophoresis through 12% acrylamide gels, and the target proteins were detected by blotting using tag-specific antibodies (SIGMA-Aldrich, Missouri, USA).

### Yeast Three-Hybrid Assays.

The cDNA fragments encoding PT2&8-CT, CK2β3 were inserted into the pBridge vector (Clontech, CA, USA) to generate fusions with GAL4DNA binding domain or Met promoter, respectively. CK2α3 was inserted into the pGADT7 vector (Clontech, CA, USA) to generate pGAD-CK2α3 to function as prey in Y3H assays. Resulting constructs vectors were co-transformed into the yeast strain AH109 and selected on dropout media lacking Leu, Met and Trp; or Leu, Met, Trp and His.

### Subcellular Localization of PT2/8 Proteins in Rice Protoplast Cells.

Isolation of rice protoplast and protoplast transient transformation were conducted as described previously (4). The wild type (Nipponbare) and mimic unphosphorylated (S512A or S517A) mutations in PT2&8 were generated with the primers by using the PT2&8-pPR3-STE plasmids as templates, all released fragments were inserted into pCAMBIA1300-GFP vector to generate fusions with GFP. Full-length CK2 α2/α3/β1/β3 fragments were cloned into the pCAMBIA35S-1300 vector (22) to generate 35S-CK2α2/α3/β1/β3 plasmids or into the pCAMBIA1300-GFP vector to generate CK2α2/α3/β1/β3-GFP. Observations were made on ZEISS Axiovert LSM 710 Laser Scanning Microscope. Protoplasts were observed under the 63× objective.

### Generation of Transgenic Plants.

Plasmids coding PT8S517-GFP and PT8S517A-GFP under control of its native promoter derived from pCAMBIA1300-PT8-GFP by replacing CAMV35S promoter with 2679 bp sequence before the ATG of PT8. For the RNAi construct, the CK2α3/β3 fragments (179 to 430 for CK2α3 and 517 to 763 for CK2β3) were cloned in both orientations in pCAMBIA35S-1300 vector, separated by the second intron of NIR1 of maize (Zea mays) to form a hairpin structure. The binary vectors and the 35S promoter driven CK2α3/β3 vectors (see above) were introduced into Agrobacterium tumefaciens strain EHA105 and transformed into the wild type rice (cv. Nipponbare) according to the method described previously (26).

### Recombinant Protein Expression.

Fragment encoding mature CK2α3/β3 and PT8-CT, as well as its alleles were cloned into expression vector pGEX-4T-1 (GE Healthcare). Fragment encoding CK2α3 was inserted into the pET30a vector (Merck) to generate the pET30-HIS-CK2α3 plasmid. The recombinant vectors were identified by sequencing. Recombinant plasmids were expressed in E. coli strain TransB(DE3)(Transgen) [F-omp T hsdSB(rB-mB-) galdcmlacY1 ahpC (DE3) gor522::Tn10 trxB(KanR, TetR); which encodes mutated thioredoxin reductase(trxB) and glutathione Reductase(gor), thus can improve the solubility of recombinant proteins] and purified using GST-affinity chromatograph on immobilized glutathione followed by competitive elution with excess reduced glutathione according to the manufacturer's instructions (GE Healthcare, NJ, USA).

### In Vitro Phosphorylation Assays.

In vitro kinase assays in solution were performed essentially as described previously (27) with a few modifications. Kinase subunits and substrate proteins were mixed with 1× kinase buffer (100 mM Tris-HCl, pH8.0, 5 mM DTT, 5 mM EGTA and 5 mM MgCl2) (New England Biolabs, MA, USA) and 1×ATP solution (100 μM ATP and 1 μCi [γ-32P]ATP) (Perkin-Elmer, Massachusetts, USA) in a total volume of 50 μL. The reactions were incubated at 30° C. for 30 min and then stopped by adding 5× loading buffer and boiling for 5 min. Products were separated by electrophoresis through 12% acrylamide gels, and the gels were stained, dried, and then visualized by exposure to X-ray films.

### In Vivo Phosphorylation Assays.

Rice seedlings (Nipponbare) and CK2α3-overexpressed/knockdown transgenic plants were grown for 7 days, and then the roots of these seedlings were harvested. The membrane protein extraction was performed as previously described (28), except that the casein was excluded from the extraction buffer. Membrane fractions were subjected to λ-phosphatase treatment as described previously (29) with a few modifications. Treatment was performed in a volume of 50 μL: the membrane fraction from the three backgrounds was added to 1×λ-phosphatase buffer and 200 units of λ-phosphatase (SIGMA-Aldrich, Missouri, USA), in a total volume of 50 μL, samples were incubated at 30° C. for 30 min. The reactions were stopped by adding 5×SDS loading buffer (Sangon, Shanghai, China) and boiled. Samples were separated in 10% Phos-tag acrylamide gels (WAKO, Osaka, Japan) and probed with PT8-specific antibody (1:500). The second antibody, goat anti-rabbit IgG peroxidase antibody (SIGMA-Aldrich, Missouri, USA), was used at 1:10,000. Detection was performed with the enhanced chemiluminescence (Pierce/Thermo Scientific, St. Leon-Rot, Germany).

### Pull-Down Assays.

PHF1N-MYC was synthesized by tobacco leaves infiltration with Agrobacterium. For in vitro binding, 20 μL of the total tobacco protein was added to 600 μL of binding buffer [50 mM Tris-HCl, pH7.5; 150 mM NaCl; 1 mM EDTA (final); 10% glycerol; 2 mM Na3VO4; 25 mM β-glycerophosphate; 10 mM NaF; 0.05-0.1% Tween 20; 1× Roche protease inhibitor; 1 mM PMSF], followed by 50 μL of glutathione-agarose beads with bound GST-PT8-CT or its alleles and was incubated at 4° C. for 3 hours. The beads were washed with binding buffer for a triple time. Bound proteins were eluted with 5×SDS loading buffer and were resolved by 12% SDSPAGE. Individual bands were detected by immunoblotting against with tag-specific antibodies. Commercial antibodies were purchased from SIGMA-Aldrich (anti-FLAG M2, 1:3,000 WB; anti-GFP, 1: 2500 WB; anti-MYC, 1:3000 WB)(St. Louis, Mo., USA), Abcam (anti-phosphoserine, 1: 250 WB) (Cambridge, UK), and GE healthcare (anti-GST, 1: 5000 WB) (NJ, USA).

### Cellular Pi and Total P Concentration Measurements.

Cellular Pi concentration and ³³P uptake analysis were conducted as previously described (4). Total P concentration in the tissues was determined as described previously (30).

### Development of PHF1 and PT8 Polyclonal Antibodies.

Polyclonal rabbit PHF1 antibody was raised against a C-terminal fragment of PHF1 corresponding to the amino acid residues 375 to 387 (C-KESPPVPEDQNPW-COOH) and affinity purified by Abmart (Shanghai, China). For an antibody against OsPT8, the synthetic peptide C-VLQVEIQEEQDKLEQMVT (positions 264-281 of OsPT8) was used to immunize rabbits. The obtained antiserum was purified through a peptide affinity column before use.

### Accession Numbers

The MSU Rice Genome Annotation Project Database accession numbers for the genes studied in this work are LOC_Os09g09000(OsPHF1), LOC_Os03g05640(OsPT2), and LOC_Os10g30790(OsPT8), LOC_Os07g02350(OsCK2 α2), LOC_Os03g10940(OsCK2 α3), LOC_Os10g41520(OsCK2β1), LOC_Os07g31280(OsC K2β3). National Center for Biotechnology Information accession numbers for the proteins are OsPH F1, NP_001059077; OsPT2, NP_001048979; OsPT8, NP 001064708; OsCK2 α2, NP_001058752; OsCK2α3, NP 001049325; OsCK2β1, NP 001065415; OsCK2β3, NP 001059693.

### Results and Discussion

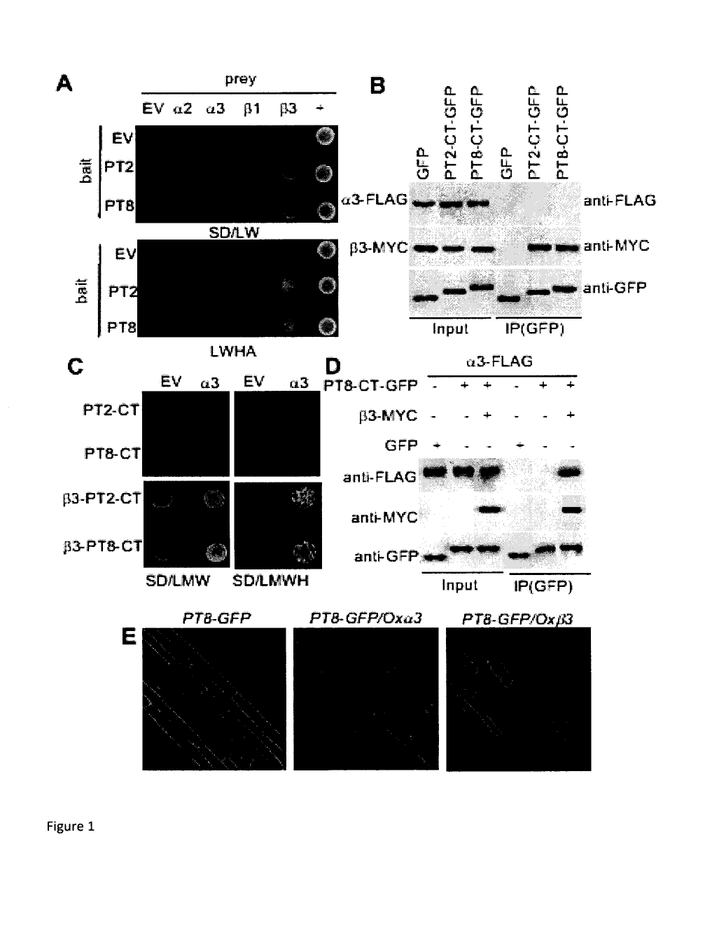

We identified a putative CK21 subunit (7, 8) interacting with a high-affinity Pi-transporter PT8 (9) was in a screen for PT8 partners of a rice root cDNA library in a yeast two-hybrid system. To confirm the initial library screening, we used another two-hybrid system and also used a second bait, PT2, a low-affinity PT for Pi translocation (10). CK2 occurs as a tetramer of two catalytic α2 subunits, α2 and α3, and two regulatory β subunits, β1 and β3 in rice (11), Yeast two-hybrid assays for interactions of the 4 components with PT2&8 indicated that only β3 interacted with PT2&PT8 in yeast cells (FIG. 1A). Previous work showed that Arabidopsis PT is phosphorylated at a hydrophilic carboxy terminal region containing two highly conserved serine amino acids (3, 4). Thus the C-termini (CT) of PT2&8 including the conserved Ser residues (Ser-507 and Ser-512 for PT2, and Ser-512 and Ser-517 for PT8) were used for in vivo interaction analysis between them and CK2β3 using co-immunoprecipitations (co-IP) assays (FIG. 1B). Results confirmed the interaction of CK2β3 with the PTs. Yeast three-hybrid assays and co-IP showed that β3 and α3 form a heterodimer interacting with the CT of PT2&8 (FIGS. 1C, D). This is agreement with a previous report indicating that CK211 subunit acts as an anchor to bind its target and interacted with a subunits to form a heteromeric holoenzyme (12).

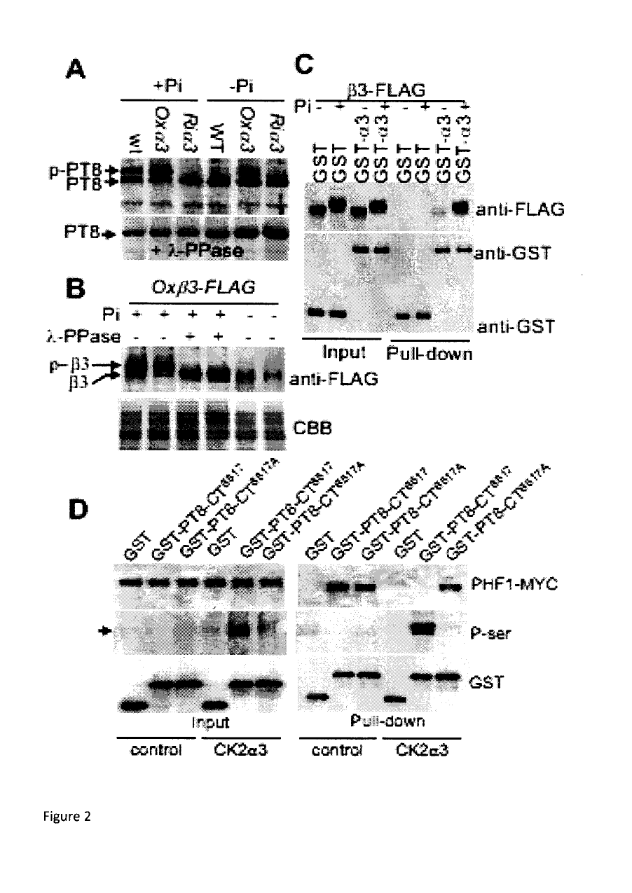

We examined the subcellular localization of PT2&8 in rice protoplasts overexpressing CK2 α3/β3 and found that PT2&8 remained retained in the ER (FIG. 1E). We also produced knockdown lines for CK2 α3 and CK2β3 using independent transgenic plants expressing RNAi constructs, to examine alterations in Pi accumulation. Independent transgenic lines grown under +P hydroponic culture (200 μM Pi) for 30 days were used for Pi concentration measurements. The knockdown transgenic plants promotes excessive Pi accumulation, especially RiCK2 α3 plants which displayed necrotic symptom on older leaf tips. The increased Pi in RiCK2 α3 and RiCK2β3 plants was accompanied by a higher Pi uptake ability in comparison with wild type (wt) plants (Nipponbare. japonica cv.). To determine whether the CK2 α3/β3 effect on PT trafficking is caused by phosphorylation of PT, we performed in vitro phosphorylation assays using recombinant GST-CK2 α3 or GST-CK2β3, and GST-PT8-CT proteins. We also tested mutant PT8-CT proteins in which Ser512 or Ser-517 was replaced with Ala (designated PT8-CTS512A and PT8-CTS517A, respectively). Results showed that the PT8-CT was phosphorylated by the catalytic subunit CK2 α3 but not by the regulatory subunit CKβ3 in vitro. Mutation of S517, but not S512, prevented phosphorylation of PT8-CT, indicating that S517 at C-terminus of PT8 is the phosphorylation site by CK2 α3. For in vivo experiments, proteins were extracted from roots of wt, CK2 α3-overexpressor (OxCK2 α3) and CK2 α3-knockdown plants (RiCK2α3) grown under Pi-supply (+P) (200 μM) and deficiency (−P) conditions and PT8 revealed using anti-PT8 antibody after immunoblotting. The phosphorylated PT8 on +P and in OxCK2 α3 plants was observed as a slower mobility band in the western blot developed with anti-PT8 antibody, and by its sensitivity to λ-phosphatase (λ-PPase) (FIG. 2A) and CK2 specific inhibitor DRB (5,6-dichloro-1-beta-D-ribofuranosylbenzimidazole) treatments. To investigate how the effect of CK2 α/β3 on PT is controlled by Pi status, we extracted the proteins from roots of 35S-CK2 α3-FLAG and 35S-CK2β3-FLAG transgenic plants grown on +P and −P. Immunoblots using anti-FLAG antibody showed no change of CK2 α3 protein level on +P and −P (FIG. S7), while autophosphorylation forms of CK2β3 under +P were observed as confirmed by λ-PPase. In contrast, P grown plants accumulated lower levels of CK2β3 which were nonphosphorylated (FIG. 2B). In line with such results, there is a report indicating that autophosphorylation of CK2β regulates its stability in mammals (13). The in vitro pull-down assays for interaction between CK2 α=3 and phosphorylated and non-phosphorylated CK2β3 showed that nonphosphorylated CK2β3 displays reduced affinity for CK2α3 (FIG. 2C). Thus −P negatively impacts both CK2β3 accumulation and interaction ability with CK2 α3. In addition, PHF1 protein level is increased greatly on −P. Thus, the reduced phosphorylation of CK2β3 and increase of PHF1 should result in enhanced ER-exit of PTs.

Because overexpression of CK2 α3/β3 leads to ER-retention of PT (FIG. 1E) Phosphorylation of PT may impair its interaction with the PT, ER-exit cofactor PHF1. To test this, we performed interaction analysis in yeast and in planta between PHF1 and wt PT8 and the mutated versions in which Ser-517 was replaced by Ala-517 or Asp-517 (designated PT8S517A or PT8S517D), that represent non-phosphorylatable PT8 or mimic phosphorylated PT8, respectively. Results showed that PHF1 interacts with wt and non-phosphorylatable PT8S517A, but not with phosphorylated-mimick PT8S517D (FIG. S8). We confirmed these findings by in vitro pull-down assays using recombinant GST-PT8-CTS517 and GST-PT8-CTS517A protein in the presence or not of CK2 α3, together with PHF1-MYC protein (FIG. 2D). In this experiment, phosphorylation of PT8-CT by CK2 α3 was monitored by phosphoserin antibody (P-ser (14). Results showed that PT8 phosphorylated in vitro by CK2α3 doesn't interact with PHF1.

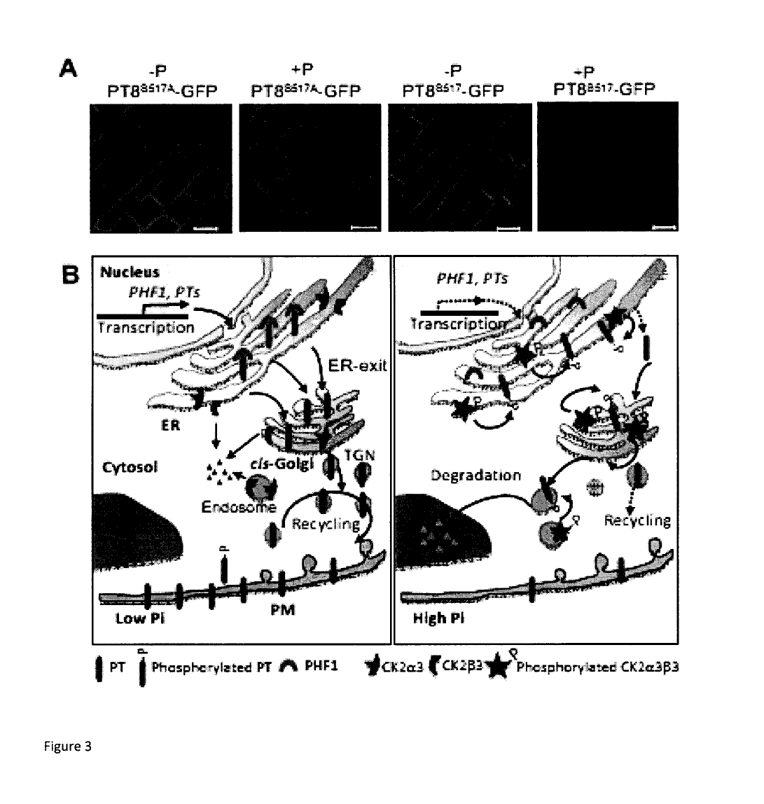

Most PTs are present in very limited amount when sufficient Pi is available in the media and the amount of PT proteins at PM is down regulated through endocytosis followed by degradation in lytic vacuoles (5). To test whether the CK2 α3/β3 is involved in recycling/degradation process of PT at the PM level, we examined whether the CK2 action extends beyond the ER. Towards this, we performed subcellular localization studies of CK2 α3 and CK2β3, using markers from different compartments (ER marker, PHF1 (4); cis-Golgi marker, GmMAN1 (15); and endosomal markers VPS29 (16) or FM4-64 (chemical dye for endocytic pathway (5). These studies showed that CK2α3 and CK2β3 were localized not only in the ER, in agreement with the regulatory role of PT phosphorylation in the negative control of its ER-exit under high Pi, but also in cis-Golgi and endosomal compartments. Next, we analyzed the stability of PT8S517-GFP (wt PT8) and PT8S517A-GFP (the non-phosphorylatable PT8) at the PM in root epidermis of plants grown under Pi-starvation (−P) and Pi-sufficient (200 μM) conditions. Results showed clear stabilization of non-phosphorylatable versus wt PT8 proteins at the PM under +P condition (FIG. 3A). The immunoblots using anti-PT8 antibody were used to detect PT8 level in PM-enriched proteins extracted from roots of the transgenic plants harboring single copy of wt PT8 (PT8S517-1) or of the non-phosphorylable PT8(PT8S517A-1) grown under different Pi levels. The results showed that PT8S517A accumulates at a significantly higher level than PT8S517 at the PM. PT8S517A accumulation is quite constant across a wide range of Pi-regimes (from 200 to 10 μM), and wt PT8 accumulation is sensitive to Pi concentration (FIG. 5). From these results, we propose a working model where CK2 α3/β3 holoenzyme acts as a key player to control ER-exit and recycling/degradation process of PTs in response to Pi status (FIG. 3B).

To determine whether the non-phosphorylatable form of PT8 may enhance Pi acquisition of plants, the wild type (wt) (XS134, a high yield japonica cultivar) and two independent transgenic lines (T2) with single copy of wt PT8 or mutant PT8S517A were used in hydroponic experiments with different Pi levels (200, 50 and 10 μM).

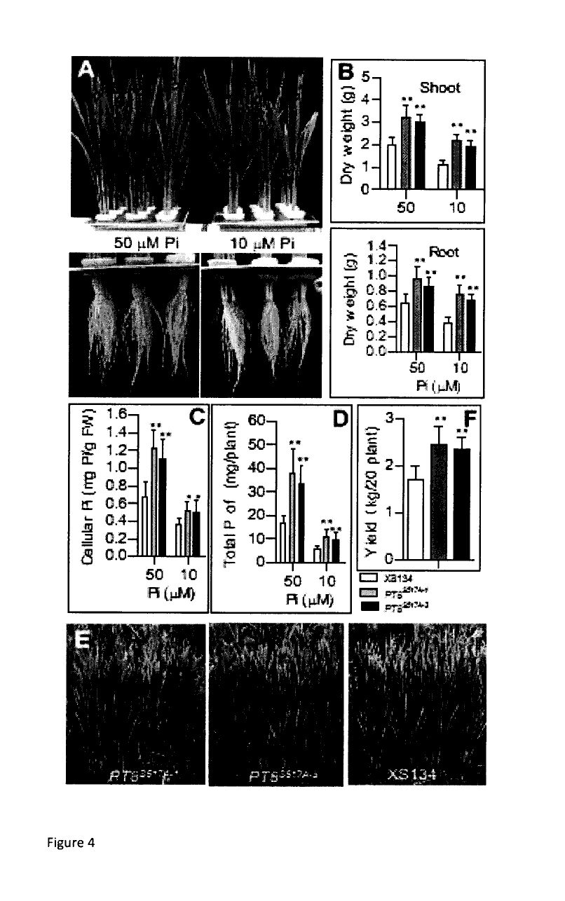

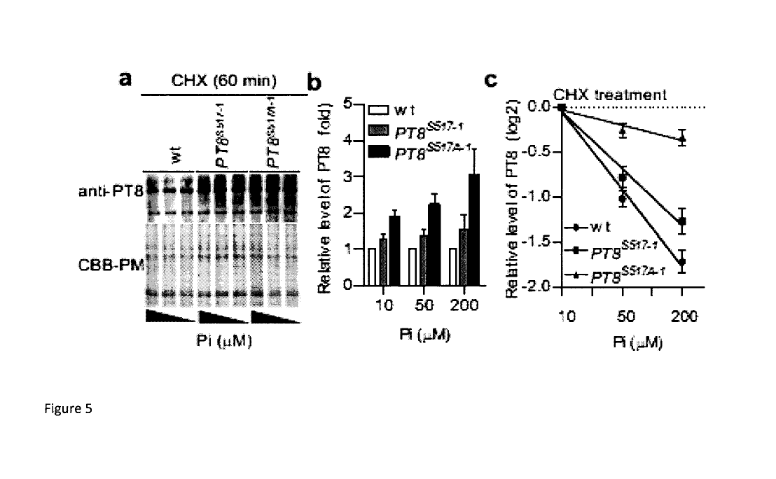

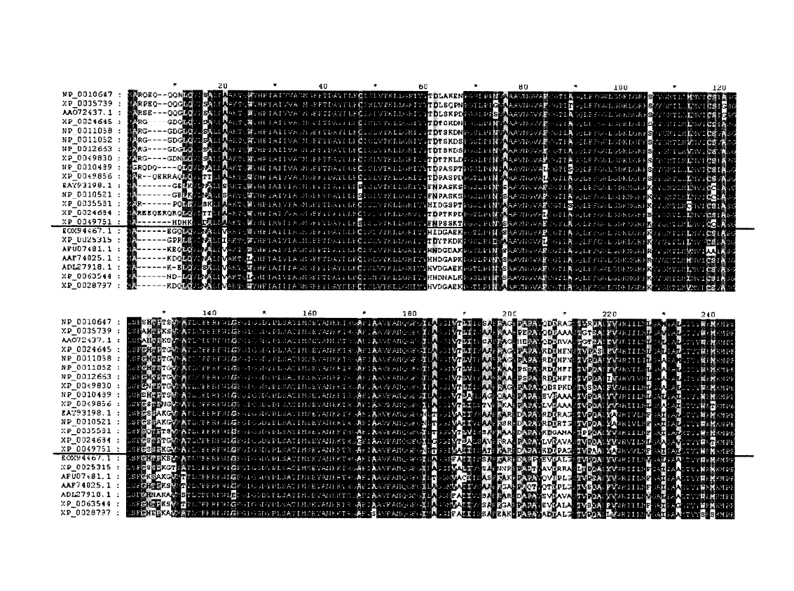

Results showed the excessive shoot Pi accumulation and Pi-toxicity symptom in older leaves of the transgenic plants with the non-phosphorylatable PT8S517A under high Pi level (200 μM). The transgenic plants expressing wt PT8 also significantly increased shoot Pi concentration in comparison with wt plants under high (200 μM) and middle (50 μM) Pi levels, but to a lower extent than PT8S517A plants. At lower Pi level (10 μM), however, only the transgenic plants expressing non-phosphorylatable PT8S517A showed significant higher Pi-acquisition ability and better growth compared to wt and the PT8S517 plants (FIG. 4A-D). In the field, plants do not face usually such very high level of Pi in soil solution. It is expected that in agriculture, plants will mostly benefit from the nonphosphorylatable PT proteins. To test this, we conducted an experiment using XS134 and two independent lines with PT8S517A in low P soil without application P-fertilizers. Field experiment showed significantly higher yield of PT8S517A plants in three randomly arranged replicates compared with XS134 (FIGS. 4E and F). The mean grain yield harvested from three replicates is about 40% higher than that of XS134 plants. These PT8S517A plants also displayed significantly higher straw dry weight, P and Zn concentrations in shoots.

Breeding crops efficiently acquiring P from native soil reserves or fertilizer sources can benefit from knowledge of mechanisms that confer enhanced uptake of this nutrient, as shown here. Indeed, we exploited our knowledge on phosphorylation control of PT activity to develop an strategy towards generating Pi-acquisition efficient rice. The recent development of efficient site directed mutagenesis methods in planta, such as those based on CRISP-Cas (19, 20), makes it feasible using this strategy with other crops, as it essentially requires altering a single codon in PT genes.

### REFERENCES

1. N. Gilbert, Environment: The disappearing nutrient. Nature 461, 716-718 (2009).
2. D. J. Conley et al., Controlling eutrophication: nitrogen and phosphorus. Science 323, 1014-1015 (2009).
3. E. Gonzalez, R. Solano, V. Rubio, A. Leyva, J. Paz-Ares, PHOSPHATE TRANSPORTER TRAFFIC FACILITATOR1 is a plant-specific SEC12-related protein that enables the endoplasmic reticulum exit of a high-affinity phosphate transporter in Arabidopsis. Plant Cell 17, 3500-3512 (2005).
4. J. Chen et al., OsPHF1 regulates the plasma membrane localization of low- and high-affinity inorganic phosphate transporters and determines inorganic phosphate uptake and translocation in rice. Plant Physiol 157, 269-278 (2011).
5. V. Bayle et al., Arabidopsis thaliana High-Affinity Phosphate Transporters Exhibit Multiple Levels of Posttranslational Regulation. Plant Cell 23, 1523-1535 (2011).
6. W. Y. Lin, T. K. Huang, T. J. Chiou. Nitrogen limitation adaptation, a target of microRNA827, mediates degradation of plasma membrane-localized phosphate transporters to maintain phosphate homeostasis in Arabidopsis. Plant Cell online (2013).
7. J. S. Rohila et al., Protein-protein interactions of tandem affinity purification-tagged protein kinases in rice. Plant J 46, 1-13 (2006).
8. F. Meggio, Pinna, L. A. One-thousand-and-one substrates of protein kinase CK2?FASEB J 17, 349-68 (2003).
9. H. Jia et al., Phosphate transporter gene, OsPht1; 8, is involved in phosphatehomeostasis in rice. Plant Physiol, 156 1164-1175 (2011).
10. P. Ai et al., Two rice phosphate transporters, OsPht1;2 and OsPht1;6, have different functions and kinetic properties in uptake and translocation. Plant J 57, 798-809 (2009).
11. X. Ding et al., A rice kinase-protein interaction map. Plant Physiol 149, 1478-1492 (2009).
12. L. A. Pinna, Protein kinase CK2: a challenge to canons. J Cell Sci 115, 3873-3878 (2002).
13. C. Zhang, G. Vilk, D. A. Canton, D. W. Litchfield, Phosphorylation regulates the stability of the regulatory CK2 beta subunit. Oncogene 21, 3754-3764 (2002).
14. J. Debreuil et al., Molecular cloning and characterization of first organic matrix protein from sclerites of red coral, Corallium rubrum. J Biol Chem 287, 19367-19376 (2012).
15. B. K. Nelson, X. Cai, A. Nebenfuhr, A multicolored set of in vivo organelle markers for co-localization studies in Arabidopsis and other plants. Plant J 51, 1126-1136 (2007).
16. Y. Jaillais et al., The retromer protein VPS29 links cell polarity and organ initiation in plants. Cell 130, 1057-1070 (2007).
17. D. L. Lopez-Arredondo, L. Herrera-Estrella, Engineering phosphorus metabolism in plants to produce a dual fertilization and weed control system. Nat Biotechnol 30, 889-893 (2012).
18. R. Gamuyao et al., The protein kinase Pstol1 from traditional rice confers tolerance of phosphorus deficiency. Nature 488, 535-539 (2012).
19. Q. W. Shan et al., Targeted genome modification of crop plants using a CRISPR-Cas system. Nat Biotechnol 31, 686-688 (2013).
20. Z. Feng et al., Efficient genome editing in plants using a CRISPR/Cas system. Cell Res 23, 1229-1232 (2013).
21. V. Bayle, L. Nussaume, R. A. Bhat, Combination of novel green fluorescent protein mutant TSapphire and DsRed variant mOrange to set up a versatile in planta FRET-FLIM assay. Plant Physiol 148, 51-60 (2008).
22. R. Yue et al., A rice stromal processing peptidase regulates chloroplast and root development. Plant Cell Physiol 51, 475-485 (2010).
23. T. Tsuge, M. Matsui, N. Wei, The subunit 1 of the COP9 signalosome suppresses gene expression through its N-terminal domain and incorporates into the complex through the PCI domain. J Mol Biol 305, 1-9 (2001).
24. V. Rubio et al., An alternative tandem affinity purification strategy applied to Arabidopsis protein complex isolation. Plant J 41, 767-778 (2005).
25. M. Dai et al., A PP6-Type Phosphatase Holoenzyme Directly Regulates PIN Phosphorylation and Auxin Efflux in Arabidopsis. Plant Cell 24, 2497-2514 (2012).
26. S. Chen et al., Distribution and characterization of over 1000 T-DNA tags in rice genome. Plant J 36, 105-113 (2003).
27. X. Meng et al., A MAPK cascade downstream of ERECTA receptor-like protein kinase regulates Arabidopsis in florescence architecture by promoting localized cell proliferation. Plant Cell 24, 4948-4960 (2012).
28. L. Abas, C. Luschnig, Maximum yields of microsomal-type membranes from small amounts of plant material without requiring ultracentrifugation. Anal Biochem 401, 217-227 (2010).
29. M. Michniewicz et al., Antagonistic regulation of PIN phosphorylation by PP2A and PINOID directs auxin flux. Cell 130, 1044-1056 (2007).
30. Z. Wu, H. Ren, S. P. McGrath, P. Wu, F. J. Zhao, Investigating the contribution of the phosphate transport pathway to arsenic accumulation in rice. Plant Physiol 157, 498-508 (2011).
31. Raghothama Ann. Rev. Plant Physiol. Plant Mol. Biol. 50: 665-693 (1999)

1-39. (canceled)

40: A transgenic monocot plant expressing a nucleic acid construct comprising a nucleic acid sequence encoding a mutant PT polypeptide comprising an amino acid modification at position S517 as set forth in SEQ ID No. 2 or of a serine at corresponding position in a sequence that is a functional variant of or homolog of SEQ ID NO. 2.

41: A transgenic monocot plant according to claim 40 wherein said modification is a substitution of the serine residue.

42: A transgenic monocot plant according to claim 41 wherein said substitution is with alanine.

43: A transgenic monocot plant according to claim 40 wherein said plant is selected from rice, wheat, barley, sorghum or maize.

44: A transgenic monocot plant according to claim 40 wherein said mutant PT polypeptide is
(a) SEQ ID NO:2 with a substitution for serine at position 517, or
(b) a homolog of SEQ ID NO: 2 and comprises an amino acid modification at the corresponding position.

45: A transgenic monocot plant according to claim 40 wherein said homolog sequence has at least 80%, at least 90%, 91%, 92%, 93%, 94%, 95%, 96%, 97%, 98%, or 99% sequence identity to SEQ ID NO. 2.

46: A transgenic monocot plant according claim 40 wherein said variant or homologous sequence is a monocot PT.

47: A transgenic monocot plant according to claim 46 wherein said plant is rice.

48: A transgenic monocot plant according to claim 40 wherein said nucleic acid construct further comprises a regulatory sequence.

49: A product derived from a plant as defined in claim 44 or from a part thereof.

50: A product derived from a plant as defined in claim 40 or from a part thereof.

51: An isolated nucleic acid encoding a mutant plant PT polypeptide comprising an amino acid substitution at position S517 as set forth in SEQ ID No. 2 or of a serine at an equivalent position in a sequence that is a functional variant of or homologous to SEQ ID NO. 2 wherein said plant is a monocot plant.

52: An isolated nucleic acid according to claim 51 wherein said modification is an amino acid substitution.

53: An isolated nucleic acid according to claim 52 wherein said substitution is with alanine.

54: An isolated nucleic acid according to claim 51 wherein said mutant PT polypeptide is a homolog of SEQ ID No. 2 and comprises an amino acid modification of a serine at a position corresponding to position S517 as set forth in SEQ ID No. 2.

55: An isolated nucleic acid according to 51 wherein said variant homolog has at least 80%, at least 90%, 91%, 92%, 93%, 94%, 95%, 96%, 97%, 98%, or 99% sequence identity to SEQ ID NO 2.

56: An isolated nucleic acid according to claim 51 wherein said homolog is from wheat, barley, sorghum or maize.

57: An isolated nucleic acid according to claim 51 which encodes a polypeptide substantially as shown in SEQ ID NO. 2 but wherein serine at position 517 in SEQ ID No. 2 is substituted.

58: A vector comprising an isolated nucleic acid according to claim 51.

59: A vector according to claim 58 further comprising a regulatory sequence.

60: A vector according to claim 58 wherein said regulatory sequence is a constitutive promoter, a strong promoter, an inducible promoter, a stress inducible promoter or a tissue specific promoter.

61: A vector according to claim 60 wherein said regulatory sequence is the CaMV35S promoter.

62: A host cell comprising a nucleic acid according to claim 51.

63: A host cell comprising vector according to claim 58.

64: A host cell according to claim 63 wherein said host cell is a bacterial or a monocot plant cell.

65: A method for increasing yield, increasing Pi uptake or zinc level, or increasing Pi use efficiency in a transgenic plant comprising introducing and expressing a nucleic acid according to claim 50 into a plant.

66: A method for increasing yield, increasing Pi uptake or zinc level, or increasing Pi use efficiency comprising introducing and expressing a vector according to claim 58 into a plant.

67: A method for increasing Pi uptake according to claim 63 wherein Pi uptake is increased under low Pi conditions.

68: A method for producing a transgenic monocot plant with increased yield comprising introducing and expressing a nucleic acid according to claim 50 into a plant.

69: A method for producing a transgenic monocot plant with increased yield comprising introducing and expressing a vector according to claim 58 into a plant.

70: A monocot plant obtained or obtainable by a method according to claim 68.

71: A monocot plant according to claim 70 wherein said plant is selected from rice, wheat, barley, sorghum, or maize

72: A method for producing a plant with increased yield comprising the steps of
a) exposing a population of plants to a mutagen and
b) identifying mutant plants in which the serine at position 517 with reference to SEQ ID No. 2 or a serine at an equivalent position in a sequence homologous to SEQ ID No. 2 is replaced by a to a non-phosphorylatable residue.

73: A method according claim 72 comprising sexually or asexually propagating or growing off-spring or descendants of the plant having increased Pi uptake and increased yield under low phosphate conditions.

74: A plant obtained or obtainable by a method of claim 72 wherein said plant is not Arabidopsis.

75: A mutant monocot plant having a mutation in a PT gene wherein said mutant PT gene encodes a mutant PT polypeptide comprising an amino acid modification at position S517 as set forth in SEQ ID No. 2 or of a serine at corresponding position in a sequence that is a functional variant of or homologous to SEQ ID NO. 2 generated by generated by mutagenesis.


SEQUENCE LISTING (40 sequences)

SEQ ID NO: 1 - DNA, 2703 bp, Oryza sativa
gtgcccagagagctcgacacaaatacagggggactcgtcttcttccccgagctttgcgagcagagtcgttcagccatggcgcggcaggagcagcagcagcacctgcaggtgctgagcgcgctggacgcggcgaagacgcagtggtaccacttcacggcgatcgtcgtcgccggcatgggcttcttcaccgacgcctacgacctcttctgcatctccctcgtcaccaagctgctcggccgcatctactacaccgacctcgccaaggagaaccccggcagcctgccgcccaacgtcgccgcggcggtgaacggagtcgcgttctgcggcacgctggcggggcagctcttcttcgggtggctcggcgacaagctcggccggaagagcgtgtacgggatgacgctgctgatgatggtcatctgctccatcgcgtcggggctctcgttctcgcacacgcccaccagcgtcatggcgacgctctgcttcttccggttctggctcggattcggcatcggcggcgactacccgctgtcggcgacgatcatgtcggagtacgccaacaagaagacccgcggcgcgttcatcgccgccgtgttcgcgatgcaggggttcggcatcctcgccggcggcatcgtcaccctcatcatctcctccgcgttccgcgccgggttcccggcgccggcgtaccaggacgaccgcgcgggctccaccgtccgccaggccgactacgtgtggcggatcatcctcatgctcggcgccatgccggcgctgctcacctactactggcggatgaagatgccggagacggcgcgctacaccgccctcgtcgccaagaacgccaagcaggccgccgccgacatgtccaaggtgctccaggtcgagatccaggaggagcaggacaagctggagcagatggtgacccggaacagcagcagcttcggcctcttctcccgccagttcgcgcgccgccacggcctccacctcgtcggcaccgccacgacatggttcctcctcgacatcgccttctacagccagaacctgttccagaaggacatcttcaccagcatcaactggatccccaaggccaagaccatgtcggcgctggaggaggtgttccgcatcgcgcgcgcccagacgctcatcgccctgtgcggcaccgtcccgggctactggttcaccgtcttcctcatcgacatcgtcggccgcttcgccatccagctgctagggtttttcatgatgaccgtgttcatgctcggcctcgccgtgccgtaccaccactggacgacgaaggggaaccacatcggcttcgtcgtcatgtacgccttcaccttcttcttcgccaacttcggccccaactccaccaccttcatcgtgccggcggagatcttcccggcgaggctgcgttccacctgccacggcatctcggcggcggcggggaaggccggcgccatcatcggatcgttcgggttcctgtacgcggcgcaggacccgcacaagcccgacgccgggtacaaacccgggatcggggtgaggaactcgctgttcgtgctcgccggatgcaacctgctcgggttcatctgcacgttcctcgtgccggagtcgaaggggaagtcgctggaggagatgtccggcgaggcggaggacgacgacgacgaggtggccgccgccggcggtggcgccgccgtgcggccgcagacggcgtagtgtatgactgcacgtgaatatagtgtaggttttacttaatttacttactgttattattattatactcctacttgtgtttgtctatgtgaaattgggaatcatgaacccatgatcatgttttgttaggttaagaaggcaaaagaaatgtgtgttaaatacttcaattatgtaaactctgtttttaagtatttggccacttgaggaataattcttgcagaccagcaatttggcacgaatacattttataattgaactaccactctaccagagtagtacactactaatttgccttagagaggacaatgagatgtctaaattttcaattatggctgtgttgagttcagcgtaaagtttagattttggttgaaattggagatgatgtgactaaaaagttgtgtgtgtatgacaggttgatgtgatggaaaaggactgaagtttggatctaaacacagcctatgaagtctaaagttattggttcaaattttgctgaaagttctgtttttcttcacaaaaagtagctttaaagttggaaatggactaactgtggagaatatatgtgaaccagatatgaaaaattgacttgcactatgatctgagtacgaagtcgaaattgagtatatttgactatgaactccatacttaggccacgtttgggggcccacagtacctaggtgacaaaaacgaaatacaacaaatttcgacgaaatttctgaccaaaggaggcctgcattgatgccgatggctcaataaagcagcagttgaattgttggacagcgtatttttctgacaaatatccggcaagtctgaagttcaggataaatagccaggcagggaagaagcgtgttcactgaattttgcaaaatttcttcagtcattcttgttgacgcggcaaaccccaatttaagccaaagtttggtaacctttttttgagtgtttggctgttaatttggggaagtgtaagtttgtttgaacagtaagtgagtatgaaaacatttattttag

SEQ ID NO: 2 - protein, 541 aa, Oryza sativa
MARQEQQQHLQVLSALDAAKTQWYHFTAIVVAGMGFFTDAYDLFCISLVTKLLGRIYYTDLAKENPGSLPPNVAAAVNGVAFCGTLAGQLFFGWLGDKLGRKSVYGMTLLMMVICSIASGLSFSHTPTSVMATLCFFRFWLGFGIGGDYPLSATIMSEYANKKTRGAFIAAVFAMQGFGILAGGIVTLIISSAFRAGFPAPAYQDDRAGSTVRQADYVWRIILMLGAMPALLTYYWRMKMPETARYTALVAKNAKQAAADMSKVLQVEIQEEQDKLEQMVTRNSSSFGLFSRQFARRHGLHLVGTATTWFLLDIAFYSQNLFQKDIFTSINWIPKAKTMSALEEVFRIARAQTLIALCGTVPGYWFTVFLIDIVGRFAIQLLGFFMMTVFMLGLAVPYHHWTTKGNHIGFVVMYAFTFFFANFGPNSTTFIVPAEIFPARLRSTCHGISAAAGKAGAIIGSFGFLYAAQDPHKPDAGYKPGIGVRNSLFVLAGCNLLGFICTFLVPESKGKSLEEMSGEAEDDDDEVAAAGGGAAVRPQTA

SEQ ID NO: 3 - DNA, 2318 bp, Oryza sativa
gtgcccagagagctcgacacaaatacagggggactcgtcttcttccccgagctttgcgagcagagtcgttcagccatggcgcggcaggagcagcagcagcacctgcaggtgctgagcgcgctggacgcggcgaagacgcagtggtaccacttcacggcgatcgtcgtcgccggcatgggcttcttcaccgacgcctacgacctcttctgcatctccctcgtcaccaagctgctcggccgcatctactacaccgacctcgccaaggagaaccccggcagcctgccgcccaacgtcgccgcggcggtgaacggagtcgcgttctgcggcacgctggcggggcagctcttcttcgggtggctcggcgacaagctcggccggaagagcgtgtacgggatgacgctgctgatgatggtcatctgctccatcgcgtcggggctctcgttctcgcacacgcccaccagcgtcatggcgacgctctgcttcttccggttctggctcggattcggcatcggcggcgactacccgctgtcggcgacgatcatgtcggagtacgccaacaagaagacccgcggcgcgttcatcgccgccgtgttcgcgatgcaggggttcggcatcctcgccggcggcatcgtcaccctcatcatctcctccgcgttccgcgccgggttcccggcgccggcgtaccaggacgaccgcgcgggctccaccgtccgccaggccgactacgtgtggcggatcatcctcatgctcggcgccatgccggcgctgctcacctactactggcggatgaagatgccggagacggcgcgctacaccgccctcgtcgccaagaacgccaagcaggccgccgccgacatgtccaaggtgctccaggtcgagatccaggaggagcaggacaagctggagcagatggtgacccggaacagcagcagcttcggcctcttctcccgccagttcgcgcgccgccacggcctccacctcgtcggcaccgccacgacatggttcctcctcgacatcgccttctacagccagaacctgttccagaaggacatcttcaccagcatcaactggatccccaaggccaagaccatgtcggcgctggaggaggtgttccgcatcgcgcgcgcccagacgctcatcgccctgtgcggcaccgtcccgggctactggttcaccgtcttcctcatcgacatcgtcggccgcttcgccatccagctgctagggtttttcatgatgaccgtgttcatgctcggcctcgccgtgccgtaccaccactggacgacgaaggggaaccacatcggcttcgtcgtcatgtacgccttcaccttcttcttcgccaacttcggccccaactccaccaccttcatcgtgccggcggagatcttcccggcgaggctgcgttccacctgccacggcatctcggcggcggcggggaaggccggcgccatcatcggatcgttcgggttcctgtacgcggcgcaggacccgcacaagcccgacgccgggtacaaacccgggatcggggtgaggaactcgctgttcgtgctcgccggatgcaacctgctcgggttcatctgcacgttcctcgtgccggagtcgaaggggaagtcgctggaggagatgtccggcgaggcggaggacgacgacgacgaggtggccgccgccggcggtggcgccgccgtgcggccgcagacggcgtagtgtatgactgcacgtgaatatagtgttgatgtgatggaaaaggactgaagtttggatctaaacacagcctatgaagtctaaagttattggttcaaattttgctgaaagttctgtttttcttcacaaaaagtagctttaaagttggaaatggactaactgtggagaatatatgtgaaccagatatgaaaaattgacttgcactatgatctgagtacgaagtcgaaattgagtatatttgactatgaactccatacttaggccacgtttgggggcccacagtacctaggtgacaaaaacgaaatacaacaaatttcgacgaaatttctgaccaaaggaggcctgcattgatgccgatggctcaataaagcagcagttgaattgttggacagcgtatttttctgacaaatatccggcaagtctgaagttcaggataaatagccaggcagggaagaagcgtgttcactgaattttgcaaaatttcttcagtcattcttgttgacgcggcaaaccccaatttaagccaaagtttggtaacctttttttgagtgtttggctgttaatttggggaagtgtaagtttgtttgaacagtaagtgagtatgaaaacatttattttag

SEQ ID NO: 4 - DNA, 1860 bp, Brachypodium distachyon
gaacccaactggtcctctcggccggcatcgtttgcatcgatcatggcgcggccggagcagcagcagggtctgcaggtgctgagcgcgctggacgcggccaagacgcagtggtaccacttcacggccatcgtggtggccggcatgggcttcttcaccgacgcctacgacctcttctgcatctccctcgtcaccaagctgctgggccgcatctactacacggacctctcccagcccaaccccggcacgctgccccccggcgtggcggcggctgtcaacggcgtggccttctgcggcacgctcaccggccagctcttcttcggctggctgggcgacaagcttggccgcaagagcgtctacgggatgacgctcctgctcatggtcatctgctccatcggctcgggcctctccttcgcgcacacccccaagagcgtcatggccacgctctgcttcttccgcttctggctcggcttcggcatcggcggcgactacccgctgtcggccaccatcatgtccgagtacgccaacaagaagacccggggcgccttcatcgccgccgtcttcgccatgcaaggcttcggcatcctggccggcggcatcgtcacgctcatcatctctgccgcgttccgtgccgcgttcccggagccggcgtaccaggacaacgccgcggcgtccacgggcacggaggccgacttcgtgtggcggatcatcctgatgctgggcgcggtgccagcgctgctgacctactactggcggatgaagatgcccgagacggcgcggtacacggcgctggtggccaagaacgccaagcaggcggcgtccgacatgtccaaggtgctgcaggtgcagatggaggacgagacggagaagctggaggagatggtgagccggggcaagaacgacttcgggctcttctccccgcagttcgcgcgccggcacgggctccacctggtgggcacggccaccacctggttcctcctggacatcgccttctacagccagaacctgttccagaaggacatcttcgcagccattaactggatccccaaggcgaaaaccatgagcgccatggacgaggtgttccgcatctcccgcgcgcagacgctcatcgcgctctgcggcaccgtgccgggctactggttcaccgtcttcctcatcgacgtcgtgggccgcttcgcgatccagctcatgggcttcttcatgatgaccgtcttcatgctgggcctcgccgtgccgtaccaccactggaccacgccagggaaccagatcgggttcgtggtcatgtacgcattcaccttcttcttcgcaaacttcgggcccaacgccaccacgttcgtcgtgccggcggagatcttcccggcaaggctgaggtccacgtgccacgggatatcggcggccgcggggaaggccggagccatgatcggggcattcgggttcctctacgcggcgcaggacccgcacaagccggaggcagggtacaagccagggatcggcgtcaggaactcgctcttcgtgctcgctggggtcaacctgttggggttcatgttcacgttcctcgtgccggaggccaacgggaagtcgctcgaggagatgtccggcgaggccgaggacaacgaggagatggccggcgccgccgtgcagccgtctcaaatggcctagtcgtcgtcgaccgtacgtacgtgacaactgctcgatcgtgttaatttggatggagatgtgttgcttctcttgtgttcatggtcaaatgatacctaccttgtttagtaatatttggttcgaacttttctttttgggcaatatatcttgttgtgatctgtaaagtttaaattagtaaagggaatcacactcccattttttgtttaa

SEQ ID NO: 5 - protein, 537 aa, Brachypodium distachyon
MARPEQQQGLQVLSALDAAKTQWYHFTAIVVAGMGFFTDAYDLFCISLVTKLLGRIYYTDLSQPNPGTLPPGVAAAVNGVAFCGTLTGQLFFGWLGDKLGRKSVYGMTLLLMVICSIGSGLSFAHTPKSVMATLCFFRFWLGFGIGGDYPLSATIMSEYANKKTRGAFIAAVFAMQGFGILAGGIVTLIISAAFRAAFPEPAYQDNAAASTGTEADFVWRIILMLGAVPALLTYYWRMKMPETARYTALVAKNAKQAASDMSKVLQVQMEDETEKLEEMVSRGKNDFGLFSPQFARRHGLHLVGTATTWFLLDIAFYSQNLFQKDIFAAINWIPKAKTMSAMDEVFRISRAQTLIALCGTVPGYWFTVFLIDVVGRFAIQLMGFFMMTVFMLGLAVPYHHWTTPGNQIGFVVMYAFTFFFANFGPNATTFVVPAEIFPARLRSTCHGISAAAGKAGAMIGAFGFLYAAQDPHKPEAGYKPGIGVRNSLFVLAGVNLLGFMFTFLVPEANGKSLEEMSGEAEDNEEMAGAAVQPSQMA

SEQ ID NO: 6 - DNA, 6561 bp, Hordeum vulgare
  misc_feature (2399)..(2399): n is a, c, g, or t
actagtgaatcaaaggttcctttagaacttgtgttttcggatgtatggggtcctggcccaatctcggttggtagacaaaagtattacgtgagctttattgatgattttagtaaattttcttggatctatttactcaaaaataagtctgatgtttttgagatgtttcatctgtttcaacagcttgttgaactcctctttaatcgcaagattttgtctatgcaaaccaattgggggtgagtaccaaaagcttaactccttctttgagtgcattggtatctccaccatgtttcctgccctcatgctcatcaacagaacgaatctgccgagcgcaaacattgccatattgttgaggttggcttgtccctgctcgctcatgcctctatgacattgaaattttgggatgaagcgtttcttacagcggtctatcttatcaaccgtgtccctagtcgagtcatccaccaccaaactccactagaacgcatgtttgatattaaaccaaactataactttcttcacatttttggttgtgcggtatggccaaatctacggcctttcaacaaacacaagctcgaattccgttccaaactgtgcgtattcataggatacagcaatctccacaaagggtacaagtgtcttgatgtttcctctggccgggtttatatttcctgcgatgttgtttttgatgatcacatcttccctttcgccaccttacatccaaatgccggcgctcaactccgcaaggagctcatacttcttccgcccaaccttctacctttgtccggtcctttaccacggggaggagtagattttgatcatatgtctatatctcataaccctggtgcaagtgtgcaggaacatacggaagaagaaatcgccgaaaacggccttgattttatgcagcaaccagatcacagcggtgcaacaaatcctggtggagatcctgatgctgattctggcgcagaatctgcctcggagtcacgcgctgcaactgcagcagacagatcctccccgggatcagcgccatcgccaggccgggcaggcggatcctctccgggtctcgcgccagcaccaggtgggtcgggcgggccctcggtaggtggatctccttcggccccgcgctagcaccaggcaggacggacgggccacatgcactgtcccccgcgtgcccctcccgacactggtcacacgcatgcacccactccggagcctccaagtggcggcactgcggctgatctgcatggcggatcttctacgactgatgcaaccgatgcttctcccgtgcatcaaactcgcctccatcaacatctctctcgaccaccgccgccaccacctgatcgactccaaaccaggtctcgtagtggcattattaaacctaaagtttataaagatggttgcgtacgctggggttctttctgttctacaggtgaaccgcaaactctggatgaggcccttagtcagtcacaatggaaggctgctatggatgaggagtattctgctcttatggagaacaacacatggcaacttgttcctcctgtcaagggcagaaatgttattggctgcaaatgggtctataaagttaaaaggaagtctaacggcaccattgacaggtacaaggctcggttggttgcaaaagggtttaagcaaaggtatggacttgactatgaggatactttcaatcatgtagttaaagttgccactatcagaattgttctttcagtagcagtatctagaagctggtgcatacggcaattagatgtgaagaacgcgtttttgcatggtgttctggaagaagaagtgtttatgaagcaacctcctggatatgagaatccacagttaccacaacatgtttgcaggcttgacaaggccttgtatggtctcaaacaagcaccaagagcttggtactataggttgtcttccaaattgcagcattgggttttatgccctcaaagggtgacacttcattgttcttttatcataggaaaggagtcactatttatatgctcatttatgttgatgatataattgtcaccaattcatgttcccaggctgttgaagctcttctcaaggatttgcgcatggattttgctctcaaagatcttggtgatctccactacttccttggcattgaggtaaaacatgtggcaagtggcattgtgctatcacgggagaaatatgtgcaggatatactccagagagcaggaatgaagaattgtaagccatctcctactcctttgtcaacttctcaaaaactgtcactttattctgggagggtacttgtgccagaagatgctaccaagtacagaagtgttgtaggagccctacaatacttaacattgactaggccatatatctcatactcagtgaataaaggatggtagttcttacatgctccaaccagnggacactttttgtcacgcccaagatgcgaccctatccttaaatttggcaccgagaagcatcatcggggatagaagcgcatctcgtcgtgtcgcatgaatggatatcggttacaagtacatggtactgaaaggaagagatatataatagaattgggcttacactcgccacaagctacatcagagtcacatcagtacattacataatcatcaagggtaagagcagggtccgactacggacgaaaacaaccgagaaaagaagaacgacgtccatccttgctatcccaggctgccggtctggaacccatcctagattgatgaagaagaagaagaagaagaagaagaagaagaagaagaagaagaagcaactccaaataaacaatccacgcgctcgcgtcaagtaacctttacatgtacttgcaactggtgttgtagtaatctgtgagccataggggactcagcaatctcatttccaaagatatcaagactagcaaagcttaatgggtgaggcatggttaagtggtgaggttgcagcagcggctaagcacatatttggtggctaaacttacgagtacaaggaataagaggggatgatctacgcataacgtagtgaactactaatgatcagatgaatgatcctgaacgcctacctacgttagacataaccccaccgtgtcctcgatcggagtaagaactcacgaaagagacagtcacggttacgcacacagttggcatattttaattaagttaacttcaagttatctagaaccagtgttaaacaaagcttccacgttgccacaattttagactatggtctaaatacatgtagctagcgggttaggtttagggacatctggaccctcagatttagatcgggtggtcaagatgattaggttagggagcccaatggacaaaccgaagacggcttgcggtaaaacagggttgatccggatacaacggtcacgaccgtatgtttcgggtaccgagaggttttcgaactaggctgcgcgtagggtcgatgcactgtgcagaggggctaggcggagattagagggaaaacgggcgacccggcgacgatttttaaaacaccgacaaccgtccgacggtagaccgaatacggtgccgctacggtcgaccgttcgggtaccagacggactccgatcgcgacgaaattcgacaggcagcctagctatatctaattacgaccgcatgccaagtttcacctcgatcagagaaagttttatgcacacttttgaaaacaagatttgacgatgtcgcgggcgcgtgcgagtgcggtcgggctcagaacggacaacgacgagaaccggcaactaacaacggatgcaagttttgaaaactggcggcaacggaatgctgatgcaatgcagatgattcgaatgatgcgatgatgatgcgacaaaagaaaatagacacacgacgaaaacggaataaaggggggatcttctggaacgtcggtcttgggctgtcacaactttgcagctgtcaaaagaatactttggtatcttcaagcaaccaagggccatggacttaagcttggtaggtcagactcaatgctagtcagtgccttctctgatgcagattgggcaggatgccctgatgacaggagatcaacacgggcaggatgcagattgctaagtcttcttaggcagcaacttagtttcctgaagtgctcgcaagcaagctactgtatccaggtcaagcacggaagctgaatataaagcactagcaaatgctaccgctgaaatcatatgggtgcagaatatgttgatagaattgggtgtttcacacccatcatcagcatctctttggtgtgataatcttggtgccacgtacttatctgctaatcctatctttcatgtcaggactaacacatatcgagattgactatcactttgttcgtgaaagagtagccagcaaacaattaaacatccggtttgtactcactggagatcaagtgacagatggttttactaaaccattgacagcacaacaactagcttcatttagacacaatcttaacttagatagtttcgatcgaggaggagtgttggaagttgtaatctacggtatgtataaaccgtatagagataacttagacttggagataagttagtttaaaccatctataccgaagagatatgacttgaagatcaatcctcgacataacaaactttgtatatcttatgctatatattaacacgcatcgcatcgcgttcgtgcaagccatacggttaacctagcttttccacgctgcggccggtctcctcctcctcgccctatttatacgagcagtaggcggcccattatttctgcaccacaacacaacaaagtcttccggccggcgggcaccgtcgtctagctctcacactcgcagcgtgccgcggccaaacgtcagtcccctgtgcagcaacagcagcagcagcatggcgcggtcggagcagcaggggctgcaggtgctgagcgcgctggacgcggccaagacgcagtggtaccacttcacggccatcgtcgtcgccggcatgggcttcttcaccgacgcctacgacctcttctgcatctccctcgtcaccaagctcctcggccgcatctactacaccgacctctccaagcccgaccccggctccctgccccccagcgtcgccgccgccgtcaacggcgtcgccttctgcggcaccctcgccggccagctcttcttcggctggctcggcgacaagatgggccgcaagagcgtctacggcatgaccctcctcctcatggtcatctgctccatcggctcgggcctctccttcgcgcacacacccaagagcgtcatggccacgctctgcttcttccgcttctggctcggcttcggcatcggcggcgactacccgctctcggccaccatcatgtccgagtacgccaacaagaagacccgcggcgcattcatcgccgccgtcttcgccatgcagggcttcggcatcctcgccggcggcatcgtcaccctcatcatctcatccgccttccgcgccgggttccacgagccggcctaccaggacgaccgcgtcgcgtccaccggcacggaggccgacttcgtgtggcgcatcatcctcatgctcggcgccctgccggccctgctcacctactactggcggatgaagatgcccgagacggcgcgctacaccgccctcgtcgccaagaacgccaagctggccgccgccgacatgtccaaggtgctgcaggtggagctggaggacgagacggagaagatggacgagatggtgagccgcggggcgaacgacttcggcctcttctcgccgcagttcgcgcggcgccacggcctccacctcgtcggcacggcgaccacgtggttcctgctggacatcgccttctacagccagaacctgttccagaaggacatcttcacgagcatcaactggatccccaaggcgcgcaccatgagcgcgcttgacgaggtgttccgcatctcccgcgcgcagacgctcatcgcgctctgcggcacagtgccgggctactggttcacggtcttcctcatcgacgtcgtcggccgcttcgccatccagctcatgggattcttcatgatgaccgtcttcatgctcggcctcgccgtgccgtaccaccactggacaacgccgggcaaccagatcggcttcgtggtcatgtacggcttcaccttcttcttcgccaacttcgggcccaacgcaaccaccttcgtcgtgccggcggagatcttcccggcgaggctgcgatcgacgtgccacgggatatcggcggccgcggggaaggccggagccatgatcggggcgttcgggttcctgtacgcggcgcaggacccgcacaagccggacgccgggtacaggcccgggatcggggtgcgcaactccctcttcgtgctcgccggggtcaacctgctggggttcatgttcaccttcctggtgccggaggccaacgggaagtcgctggaggagatgtccggcgaggcacaggacaacgagaacgaggaccaggcacgaaccgccgccgtgcagccgtccatggcctaggacaactcgtgcgtgctagctattgcagctgcaggctgttgagttggtcgaagatccttaatttggtttttgtgatacatataaacgcttaaactactactagt

SEQ ID NO: 7 - protein, 538 aa, Hordeum vulgare
MARSEQQGLQVLSALDAAKTQWYHFTAIVVAGMGFFTDAYDLFCISLVTKLLGRIYYTDLSKPDPGSLPPSVAAAVNGVAFCGTLAGQLFFGWLGDKMGRKSVYGMTLLLMVICSIGSGLSFAHTPKSVMATLCFFRFWLGFGIGGDYPLSATIMSEYANKKTRGAFIAAVFAMQGFGILAGGIVTLIISSAFRAGFHEPAYQDDRVASTGTEADFVWRIILMLGALPALLTYYWRMKMPETARYTALVAKNAKLAAADMSKVLQVELEDETEKMDEMVSRGANDFGLFSPQFARRHGLHLVGTATTWFLLDIAFYSQNLFQKDIFTSINWIPKARTMSALDEVFRISRAQTLIALCGTVPGYWFTVFLIDVVGRFAIQLMGFFMMTVFMLGLAVPYHHWTTPGNQIGFVVMYGFTFFFANFGPNATTFVVPAEIFPARLRSTCHGISAAAGKAGAMIGAFGFLYAAQDPHKPDAGYRPGIGVRNSLFVLAGVNLLGFMFTFLVPEANGKSLEEMSGEAQDNENEDQARTAAVQPSMA

SEQ ID NO: 8 - DNA, 2025 bp, Sorghum bicolor
ccatttgtgcacacccacaagcttcaagctccctccaggcatctcccaaatccccccagccgaacaaacaaacttggacaggggcagctagcatccatccatccgccatggcgcgcgggggagacggcctgcaggtgctcagcgcgctggacgcggccaagacgcagtggtaccacttcacggccatcatcgtggccggcatgggcttcttcaccgacgcctacgacctcttctgcatctccctcgtcaccaagctgctgggccgcatctactacacggacaccagcaaggacaaccccggctcgctccctcccaacgtcgccgccgcggtcaacggcgtcgccttctgcggcacgctcgccggccagctcttcttcggctggctcggcgacaagctcggccgcaagagcgtctacgggatgacgctcatgctcatggtcatctgctccatcgcgtcgggcctctccttcggccacacccccacgggtgtcatggccacgctctgcttcttccgcttctggctcgggttcggcatcggcggcgactacccgctgtccgccaccatcatgtccgagtacgccaacaagaagacccgcggcgccttcatcgccgccgtcttcgccatgcagggcttcggcatcctcgccggtggcattgtcacgctcatcatctccgccgcgttccgcgccgggtaccctgccccggcgtacaaggacgaccacttcaactccaccgtgccgcagtccgacttcgtgtggcgcatcatcctcatgctcggcgccttgccggcgctgctcacctactactggcggatgaagatgcccgagacggcgcgctacaccgcgctggtggccaagaacgccaagcaggccgcggccgacatgtccaaggtgctccacacggagatcgtcgacgagcaggagaagctggaccagatggtcaccgccgagagcaacaccttcggcctcttctccagggagttcgcgcgccgccacggcctccacctcgtcggcaccgccaccacctggttcctgctcgacatcgccttctacagccagaacctgttccagaaggacatcttcacggccatcaactggatccccaaggccaacaccatgagcgcgctcgaggaggtgttccgcatctcccgcgcgcagacgctcatcgcgctctgcggcaccgtcccggggtactggttcaccgtcgcgctcatcgacgtcgtcggacgattcgccatccagctgctcggattcttcatgatgaccgtcttcatgctcggcctcgccatcccctaccaccactggaccaccgccggcaaccacatcggattcgtcgtcatgtacggcttcaccttcttcttcgcaaacttcgggcccaacagcaccaccttcatcgtgccggctgagatcttcccggcgcggctgcgctccacctgccacggcatctccgccgcctcggggaaggccggagccatcatcggcgccttcgggttcctgtacgcggcgcagaaccaggacaagagcaaggcggacgccgggtaccccgcgggcatcggcgtgcgcaactcgctcttcgtcctcgcgggctgcaacatgctcggattcgtcctcactttcctcgtgccggagtccaagggcaagtcgctggaggagatgtcaggtgaggctgacgacgccgaggaggaggccgtcggcgcccgcgcggtgcggccgtcggagacccagatggtatagaatcgaacgcgttcacacgggagctggttcttcttcttcttcttcttcttcctgcacctgcacatggggagacttgtgtaggcgcttcagttcaattgtgtttttgtttctccatgtatgcaagtcaagtcgcacctctgtttcgctgtccaggagtagttcgtatgtggtgcaggtgcatgtgtgttatgtacttattataggcgggaaagtaagtgaacttacactcgtatacccaaccttttactggtgtgaaaagttatgctgaaaatattattcgctgatttattat

SEQ ID NO: 9 - protein, 541 aa, Sorghum bicolor
MARGGDGLQVLSALDAAKTQWYHFTAIIVAGMGFFTDAYDLFCISLVTKLLGRIYYTDTSKDNPGSLPPNVAAAVNGVAFCGTLAGQLFFGWLGDKLGRKSVYGMTLMLMVICSIASGLSFGHTPTGVMATLCFFRFWLGFGIGGDYPLSATIMSEYANKKTRGAFIAAVFAMQGFGILAGGIVTLIISAAFRAGYPAPAYKDDHFNSTVPQSDFVWRIILMLGALPALLTYYWRMKMPETARYTALVAKNAKQAAADMSKVLHTEIVDEQEKLDQMVTAESNTFGLFSREFARRHGLHLVGTATTWFLLDIAFYSQNLFQKDIFTAINWIPKANTMSALEEVFRISRAQTLIALCGTVPGYWFTVALIDVVGRFAIQLLGFFMMTVFMLGLAIPYHHWTTAGNHIGFVVMYGFTFFFANFGPNSTTFIVPAEIFPARLRSTCHGISAASGKAGAIIGAFGFLYAAQNQDKSKADAGYPAGIGVRNSLFVLAGCNMLGFVLTFLVPESKGKSLEEMSGEADDAEEEAVGARAVRPSETQMV

SEQ ID NO: 10 - DNA, 1939 bp, Zea mays
caacaagctaggcagctgcaagcatctcccaaatcccagcctgctgccccgcgcgaacacacttctcttgtcgttgcattgcagggcagctagccggctacagcatccgccatggcgcgcggcggggacggcctgcaggtgctcagcgcgctggacgcggcgaagacgcagtggtaccacttcacggccatcatcgtcgccggcatgggcttcttcacggacgcctacgacctcttctgcatctccctcgtcaccaagctgctggggcgcatctactacacggacaccagcaaggacaacccgggctcgctcccgcccaacgtcgccgcggcggtcaacggcgtcgccttctgcggcacgctggccggccagctcttcttcggctggctcggggacaagctcgggcgcaagagcgtgtacgggatgacgctcatgctcatggtcatctgctccgtcgcgtcgggcctctcgttcggccacacgcccacgggggtcatggccacgctctgcttcttccgcttctggctcgggttcggcatcggcggggactacccgctgtcggcgaccatcatgtccgagtacgccaacaagaagacccgcggcgccttcatcgcggccgtcttcgccatgcagggcttcggcatcctcgccggcggcattgtcacgctcgtcatctccgccgccttccgcgcggggtacccggccccggcgtacagggacgaccacttcaactccaccgtgccgcaggccgactacgtgtggcgcatcatcctcatcctgggcgccgcgccggcgatgctcacctactactggcggatgaagatgcccgagacggcgcgctacaccgcgctcgtggccaagaacgccaagcaggccgcggccgacatgtccagggtgctccagacggagatcgtcgacgagcaggagaagctggacgagatggtcaccgccgagagcaacaccttcggcctcttctccagggagttcgcgcgccgccacgggctccacctcgtcggcacctccaccacgtggttcctgctcgacatcgccttctacagccagaacctgttccagaaggacatcttcaccagcatcaactggatccccaaggccaacaccatgagcgcgctggaggaggtgttccgcatctcccgcgcccagacgctcatcgcgctctgcggcaccgtcccgggctactggttcaccgtcgcgctcatcgacgtcgtcggccgcttcgccatccagctgctcggcttcttcatgatgaccgtcttcatgctcggcctcgccatcccctaccaccactggaccacgcccggcaaccacatcggcttcgtcgtcatgtacgccttcaccttcttcttcgcaaacttcggccccaacagcaccaccttcatcgtgccggccgagatcttcccggcgcggctgcggtccacatgccacggcatctccgccgcctcggggaaggccggcgccatcatcggggcctttgggttcctgtacgcggcgcagaaccaggacaagagcaaggcggacgccgggtaccccgcgggcatcggcgtacgcaattcgctcttcgtcctcgctgcctccaacttgcttggctttatcctcaccttcctcgtgccggagtccaagggtaagtcgctcgaggagatgtccggggaggctgacgacgccgaggacgacgccgtcggcacccgcgcggtgcggccgtcggggacccagatggtgtagaatcgaacatggcgacgcgtgcacacgggtcctccatcctggggcccgactgggaagacaaggcgcgccggttcaagcctgccgatgctttgctgtgcagaagtagttcgtacaggtgcatgtgtgttataggcgggaaagtaagtgaacttacacgcttgtatttattatatccaaccttacgtacaaaaaaaaaaaaaaa

SEQ ID NO: 11 - protein, 541 aa, Zea mays
MARGGDGLQVLSALDAAKTQWYHFTAIIVAGMGFFTDAYDLFCISLVTKLLGRIYYTDTSKDNPGSLPPNVAAAVNGVAFCGTLAGQLFFGWLGDKLGRKSVYGMTLMLMVICSVASGLSFGHTPTGVMATLCFFRFWLGFGIGGDYPLSATIMSEYANKKTRGAFIAAVFAMQGFGILAGGIVTLVISAAFRAGYPAPAYRDDHFNSTVPQADYVWRIILILGAAPAMLTYYWRMKMPETARYTALVAKNAKQAAADMSRVLQTEIVDEQEKLDEMVTAESNTFGLFSREFARRHGLHLVGTSTTWFLLDIAFYSQNLFQKDIFTSINWIPKANTMSALEEVFRISRAQTLIALCGTVPGYWFTVALIDVVGRFAIQLLGFFMMTVFMLGLAIPYHHWTTPGNHIGFVVMYAFTFFFANFGPNSTTFIVPAEIFPARLRSTCHGISAASGKAGAIIGAFGFLYAAQNQDKSKADAGYPAGIGVRNSLFVLAASNLLGFILTFLVPESKGKSLEEMSGEADDAEDDAVGTRAVRPSGTQMV

SEQ ID NO: 12 - DNA, 1770 bp, Zea mays
ttctctggtcgtcgcatcgcagggcagctagctaggtagctaacatccgccatggcgcgcgggggagacggcctgcaggtgctgagcgcgctggacgcggcgaagacgcagtggtaccacttcacggccatcatcgtcgccggcatgggcttcttcacggacgcctacgacctcttctgcatctccctcgtcaccaagctgctgggccgcatctactacacggacaccagcaaggacagccccgggtcgctgccgcccaacgtcgcggcggcggtcaacggcgtggccttctgcggcacgctggccgggcagctcttcttcggctggctgggcgacaagctggggcgcaagagcgtgtacgggatgacgctcatggtcatggtcatctgctccgtcgcgtcgggcctctcgttcggccacacccccacgggggtcatggccacgctctgcttcttccgcttctggctcggcttcggcatcggcggcgactacccgctgtcggccaccatcatgtccgagtacgccaacaagaggacccgcggcgccttcatcgcggccgtcttcgccatgcagggcttcggcatcctcgccggcggcatcgtcacgctcgtcatctccgccgccttccgcgcggcgtacccgtccccggcgtacagggacgaccacttcacctccaccgtgccgcaggccgacttcgtgtggcgggtcatcctcatgctcggcgccgcgccggcgctgctcacctactactggcggatgaagatgcccgagacggcgcggtacacggcgctggtggccaagaacgccaagcaggccgcggccgacatgtccaaggtgctgcagactgagatcgtggacgagcaggagaagctggacgccgccgagggcgccaacagcttcggcctcttctccagggagttcgcgcgccgccacggcctccacctcgtgggcaccgccaccacctggttcctgctcgacatcgccttctacagccagaacctgttccagaaggacatcttcaccagcatcaactggatccccaaggccaacaccatgagcgcgctggaggaggtgtaccgcatctcccgcgcgcagaccctcatcgcgctctgcggcacagtcccgggctactggttcaccgtcgcgctcatcgacgtcgtcggccgcttcgccatacagctgctgggcttcttcatgatgaccgtcttcatgctcggcctcgccatcccctaccaccactggaccacgccgggcaaccacatcggcttcgtcgtcatgtacgccttcaccttcttcttcgccaacttcgggcccaacagcaccaccttcatcgtgcccgccgagatcttcccggcgcgcctgcgctccacctgccacggcatctccgccgcctcggggaaggccggggccatcatcggcgcgttcggcttcctgtacgcggcgcagaaccaggacaggagcaagacggacgccggctaccccgcgggcatcggcgtgcgcaactcgctcttcgtcctcgccgccagcaacatgctcggcttcgtcctcacgttcctcgtgccggagtccaggggcaagtcgctcgaggagatgtccggtgaggctgaagactcagaggaggagcccgtcggcgcccgtgcggtgcggccgtcggagacccagatggtgtagagaatcgatcgatcgacgcgtgttccttcctgcactgcacatggtgggctatcatgtcctcaattgtttttttccacgttaaagtcaaccctggctgtg

SEQ ID NO: 13 - protein, 539 aa, Zea mays
MARGGDGLQVLSALDAAKTQWYHFTAIIVAGMGFFTDAYDLFCISLVTKLLGRIYYTDTSKDSPGSLPPNVAAAVNGVAFCGTLAGQLFFGWLGDKLGRKSVYGMTLMVMVICSVASGLSFGHTPTGVMATLCFFRFWLGFGIGGDYPLSATIMSEYANKRTRGAFIAAVFAMQGFGILAGGIVTLVISAAFRAAYPSPAYRDDHFTSTVPQADFVWRVILMLGAAPALLTYYWRMKMPETARYTALVAKNAKQAAADMSKVLQTEIVDEQEKLDAAEGANSFGLFSREFARRHGLHLVGTATTWFLLDIAFYSQNLFQKDIFTSINWIPKANTMSALEEVYRISRAQTLIALCGTVPGYWFTVALIDVVGRFAIQLLGFFMMTVFMLGLAIPYHHWTTPGNHIGFVVMYAFTFFFANFGPNSTTFIVPAEIFPARLRSTCHGISAASGKAGAIIGAFGFLYAAQNQDRSKTDAGYPAGIGVRNSLFVLAASNMLGFVLTFLVPESRGKSLEEMSGEAEDSEEEPVGARAVRPSETQMV

SEQ ID NO: 14 - DNA, 1795 bp, Zea mays
cccgaagaacacccttctctggtcgtcgcatcgcagggcagctagctaggtagctaacatccgccatggcgcgcgggggagacggcctgcaggtgctgagcgcgctggacgcggcgaagacgcagtggtaccacttcacggccatcatcgtcgccggcatgggcttcttcacggacgcctacgacctcttctgcatctccctcgtcaccaagctgctgggccgcatctactacacggacaccagcaaggacagccccgggtcgctgccgcccaacgtcgcggcggcggtcaacggcgtggccttctgcggcacgctggcggggcagctcttcttcggctggctgggcgacaagctggggcgcaagagcgtgtacgggatgacgctcatggtcatggtcatctgctccgtcgcgtcgggcctctcgttcggccacacccccacgggggtcatggccacgctctgcttcttccgcttctggctcggcttcggcatcggcggcgactacccgctgtcggccaccatcatgtccgagtacgccaacaagaggacccgcggcgccttcatcgccgccgtcttcgccatgcagggcttcggcatcctcgccggcggcatcgtcacgctcgtcatctccgccgccttccgcgcggcgtacccgtccccggcgtacagggacgaccacttcacctccaccgtgccgcaggccgacatcgtgtggcgggtcatcgtcatgctcggcgccgcgccggcgctgctcacctactactggcggatgaagatgcccgagacggcgcggtacacggcgctggtggccaagaacgccaagcaggccgccgccgacatgtccaaggtgctgcacacggagatcgtggacgagcaggagaagctggacgccgccgagggcgccaacagcttcggcctcttctccagggagttcgcgcgccgccacggcctccacctcgtcggcaccgccaccacctggttcctgctcgacatcgccttctacagccagaacctgttccagaaggacatcttcaccagcatcaactggatccccaaggccaacaccatgagcgcgctggaggaggtgtaccgcatctcccgcgcgcagaccctcatcgcgctctgcggcacagtcccgggctactggttcaccgtcgcgctcatcgacgtcgtcggccgcttcgccatacagctgctgggcttcttcatgatgaccgtcttcatgctcggcctcgccatcccctaccaccactggaccacgccgggcaaccacatcggcttcgtcgtcatgtacgccttcaccttcttcttcgccaacttcgggcccaacagcaccaccttcatcgtgcccgccgagatcttcccggcgcgcctgcgctccacctgccacggcatctccgccgcctcggggaaggccggggccatcatcggcgcgttcggcttcctgtacgcggcgcagaaccaggacaggagcaagacggacgccggctaccccgcgggcatcggcgtgcgcaactcgctcttcgtcctcgccgccagcaacatgctcggcttcgtcctcacgttcctcgtgccggagtccaagggcaagtcgctcgaggagatgtccggtgaggctgaagactcagaggaggagcccgtcggcgcccgtgcggtgcggccgtcggagacccagatggtgtagagaatcgatcgatcgacgcgtgttccttcctgcactgcacatggtgggctatcatgtcctcaattgtttttttccacgttaaagtcaaccctggctgtgttttgatgtgc

SEQ ID NO: 15 - protein, 539 aa, Zea mays
MARGGDGLQVLSALDAAKTQWYHFTAIIVAGMGFFTDAYDLFCISLVTKLLGRIYYTDTSKDSPGSLPPNVAAAVNGVAFCGTLAGQLFFGWLGDKLGRKSVYGMTLMVMVICSVASGLSFGHTPTGVMATLCFFRFWLGFGIGGDYPLSATIMSEYANKRTRGAFIAAVFAMQGFGILAGGIVTLVISAAFRAAYPSPAYRDDHFTSTVPQADIVWRVIVMLGAAPALLTYYWRMKMPETARYTALVAKNAKQAAADMSKVLHTEIVDEQEKLDAAEGANSFGLFSREFARRHGLHLVGTATTWFLLDIAFYSQNLFQKDIFTSINWIPKANTMSALEEVYRISRAQTLIALCGTVPGYWFTVALIDVVGRFAIQLLGFFMMTVFMLGLAIPYHHWTTPGNHIGFVVMYAFTFFFANFGPNSTTFIVPAEIFPARLRSTCHGISAASGKAGAIIGAFGFLYAAQNQDRSKTDAGYPAGIGVRNSLFVLAASNMLGFVLTFLVPESKGKSLEEMSGEAEDSEEEPVGARAVRPSETQMV

SEQ ID NO: 16 - DNA, 1883 bp, Setaria italica
cagcgcaaggcacacctcccaaacacagcagagccggatcgatccggcggccctcttgccagtgcgcggcggcggcggctagttctcggcgggcgccatggcgcgtgggggcgacaacctgcaggtgctgagcgcgctggacgcggccaagacgcagtggtaccacttcacggccatcatcgtcgccggcatgggcttcttcaccgacgcctacgacctcttctgcatctccctcgtcaccaagctgctcggccgcatctactacaccgacaccaccaagctcgacccgggctcgctgccgcccaacgtcgccgccgccgtcaacggcgtcgccttctgcggcacgctggcgggccagctcttcttcggctggctcggcgacaagctcggccgcaagagcgtctacgggatgacgctcatgctcatggtgctctgctccatcgcgtccgggctctcgttcggcaacacccccacgggggtcatggccacgctctgcttcttccgattctggctcggcttcggcatcggcggcgactacccgctctcggcgaccatcatgtccgagtacgccaacaagcgcacccgcggtgccttcatcgcggccgtcttcgccatgcaggggttcggcatcctcgccggcggcatcgtcacgctcatcatctccgcggcgttccgcgccgggtaccctgccccggcgtaccaggacagccccaaggactccaccgtgtcgcaggccgacttcgtgtggcgcatcatcctcatgctcggcgccgcgccggccctgctcacctactactggcggatgaagatgcccgagacggcgcgctacaccgcgctcgtcgccaagaacgccaagcaggccgccgccgacatgtccaaggtcctccagacggagatcgtcgacgagcaggagaagctcgacacgatggtcacctccacgggcaacagcttcggcctcttctccagggagttcgcgcgccgccacgggctccacctcctcggcaccgccagcacgtggttcctgctcgacatcgccttctacagccagaacctgttccagaaggacatcttcaccagcatcaactggatccccaaggcccgcaccatgagcgcgctcgaggaggtgttccggatctcccgcgcccagacgctcatcgcgctctgcggcaccgtcccgggctactggttcaccgtcgccctcatcgacgtcgtcggacgcttcaccatccagctgctggggttcttcatgatgaccgtcttcatgctcggcctcgccgtgccgtaccaccactggacgacgccgggcaaccacatcggcttcgtcgtcatgtacgccttcaccttcttcttcgccaacttcgggcccaacagcacgacctttatcgtgcccgccgagatcttcccggcgcggctgcggtcgacgtgccacggcatctccgccgccgcggggaaggccggcgccatcatcggggcgttcgggttcctgtacgcggcgcagaaccaggacaagagcaaggtggaccacgggtaccccgcgggcatcggcgtccgcaactcgctcttcgtgctcgcaggggtcaacatgctcggcttcatactcacgttcctcgtgccggagtccaaggggaagtcgctcgaggagatgtccggcgaggccgacgacggcgaggaggaggccgtcggcggccgcgcggtgcggccgtcccagacccagatggtgtagtatgaccgtccgtggtgattggtgatacgtgtaggccggttcacttgttttcgttttccatgtagaaagtcaaacctgctgtttcacatgggcatctgttatttttatctctatataaaatataaaaaagaaaatatcaagtacacaaatacattggtga

SEQ ID NO: 17 - protein, 541 aa, Setaria italica
MARGGDNLQVLSALDAAKTQWYHFTAIIVAGMGFFTDAYDLFCISLVTKLLGRIYYTDTTKLDPGSLPPNVAAAVNGVAFCGTLAGQLFFGWLGDKLGRKSVYGMTLMLMVLCSIASGLSFGNTPTGVMATLCFFRFWLGFGIGGDYPLSATIMSEYANKRTRGAFIAAVFAMQGFGILAGGIVTLIISAAFRAGYPAPAYQDSPKDSTVSQADFVWRIILMLGAAPALLTYYWRMKMPETARYTALVAKNAKQAAADMSKVLQTEIVDEQEKLDTMVTSTGNSFGLFSREFARRHGLHLLGTASTWFLLDIAFYSQNLFQKDIFTSINWIPKARTMSALEEVFRISRAQTLIALCGTVPGYWFTVALIDVVGRFTIQLLGFFMMTVFMLGLAVPYHHWTTPGNHIGFVVMYAFTFFFANFGPNSTTFIVPAEIFPARLRSTCHGISAAAGKAGAIIGAFGFLYAAQNQDKSKVDHGYPAGIGVRNSLFVLAGVNMLGFILTFLVPESKGKSLEEMSGEADDGEEEAVGGRAVRPSQTQMV

SEQ ID NO: 18 - DNA, 1801 bp, Oryza sativa
atctgttataaccatcgcgttggatcgtagcagcagccgccgacccaaacgcaaacgcaaacgcgacgccatgggaaggcaggaccagcagctgcaggtgctgaacgcgctcgacgcggccaagacgcaatggtaccacttcacggcgatcatcgtcgccggcatggggttcttcaccgacgcctacgacctcttctgcatctcgctcgtcaccaagcttctcggccgcatctactacaccgaccccgccagccccacccccggctcgctgccgcccaacatcgccgccgcggtgaatggcgtcgcgctctgcggcaccctctccggccagctcttcttcggatggctcggcgacaagctcggccgcaagagcgtctacgggatgacgctgctgctcatggtgatttgctccatcgcctcagggctctccttctcgcacacgccgacgagcgtcatggccacgctctgcttcttccgcttctggctcggcttcggcatcggcggtgactacccgctgagcgccaccatcatgtccgagtacgccaacaagaagacccgcggcgcgttcatcgccgccgtcttcgccatgcaggggttcggcatcctcgccggcggcgttgtcacgctcgccatgtccgcggggttccaggccgcgttcccggccccagcgtacgaggtcaatgccgctgcgtccaccgtgccgcaggccgactacgtgtggcgcatcatcctgatgctcggtgcgctgccggccatactgacgtactactggcggatgaagatgccggagacggcgcggtacacggcgctcgtcgccaaggacgcgaagcaggcgtcgtcggacatggccaaggtgctgcaggtggaaatcgaggtggaggaggagaagctccaggacatcacgaggggcagggactacggcctcttctcggcgcggttcgccaagcgccatggcgcgcacctcctgggcacggcggcgacgtggttcctcgtcgacgtcgcgtactacagccagaacctgttccagaaggacatcttcaccagcatccactggatccccaaggcgcgcaccatgagcgagctcgaggaggtgttccgcatctcccgcgcgcagacgctcatcgcgctctgcggcaccgtgccgggctactggttcaccgtcttcctcatcgacatcatcggccgcttcaagatccagctcctcggcttcgccgggatgacggcgttcatgctcggcctcgccatcccgtaccaccactggaccatgcctggcaaccaggtcatcttcgtcttcctctacggcttcaccttcttcttcgccaactttgggccgaacgcgacgacgttcatcgtgccggccgagatcttcccggcgcgtctccggtcaacctgccacggcatctccgccgcgtccggcaaggccggcgcgatcatcggagcattcggtttcctctacgcggcgcagccacaggacaaggcgcatgtcgacgccggctacaaacctgggattggcgtgcggaacgcgctcttcgtgctcgccgggtgcaacctcgttgggttcctcatgacatggatgctcgtgccggaatcgaaagggaagtcgctggaggagatgtccggcgaggccgacgacgaggaagcttctgccaacggcggtgccaccgccgtcaactcgtccggagttgagatggtgtaatccttcaggacgcaacgagatgacgaacacttgcatgcgaagctcgtacttgtagcgtgataggaaatgttatacttatatttattagatcgtactcctactagt

SEQ ID NO: 19 - protein, 541 aa, Oryza sativa
MGRQDQQLQVLNALDAAKTQWYHFTAIIVAGMGFFTDAYDLFCISLVTKLLGRIYYTDPASPTPGSLPPNIAAAVNGVALCGTLSGQLFFGWLGDKLGRKSVYGMTLLLMVICSIASGLSFSHTPTSVMATLCFFRFWLGFGIGGDYPLSATIMSEYANKKTRGAFIAAVFAMQGFGILAGGVVTLAMSAGFQAAFPAPAYEVNAAASTVPQADYVWRIILMLGALPAILTYYWRMKMPETARYTALVAKDAKQASSDMAKVLQVEIEVEEEKLQDITRGRDYGLFSARFAKRHGAHLLGTAATWFLVDVAYYSQNLFQKDIFTSIHWIPKARTMSELEEVFRISRAQTLIALCGTVPGYWFTVFLIDIIGRFKIQLLGFAGMTAFMLGLAIPYHHWTMPGNQVIFVFLYGFTFFFANFGPNATTFIVPAEIFPARLRSTCHGISAASGKAGAIIGAFGFLYAAQPQDKAHVDAGYKPGIGVRNALFVLAGCNLVGFLMTWMLVPESKGKSLEEMSGEADDEEASANGGATAVNSSGVEMV

SEQ ID NO: 20 - DNA, 1894 bp, Setaria italica
cgtaacaacctcggctccccccttccgatctactcctacatttgaggcgttggagttcttggcggcggcggcggcggcggcagcagcagcagtgcgtcgagaccgcgcggcaccatggcgaggcaggagcggcgggcgcagctccaggtgctgaccacgctcgacgccgccaagacgcagtggtaccacttcacggcgatcgtcgtcgcgggcatgggcttcttcaccgacgcctacgacctcttctgcatctcgctcgtcaccaagctcctcggccgcatctactacaccgaccccgccagccccgaccccggcacgctgccgcccaacgtcgccgccgcggtgaacggcgtcgcgctctgcggcacgctcgcggggcagctcttcttcggctggctcggcgacaagctcggccgcaagagcgtctacggcatgacgctgctgctcatggtgatctgctccgtcgcgtccgggctctcgttcgggagcacccccaacggcgtcatggccacgctctgcttcttccgcttctggctcggcttcggcatcggcggcgactacccgctcagcgccaccatcatgtctgagtacgccaacaagaagacccgcggtgccttcatcgccgccgtgttcgccatgcagggcttcggcatcctcgccggcggcatcgtcacgctcatcctctccacggtgttccgcaaggccttcccggcgccggcgtacctggttgatgccgcggcgtccaccgtcccgcaggccgactacgtgtggcgcatcatcctcatgctcggcgcggcgcctgcgatcctgacctactactggcggacgaagatgcccgagacggcgcggtacaccgcgctggtcgccaagaacgccaagcaggccgccgcggacatgtccaaggtgctgcaggtggagatcgatgcggagtcggagaagctggacgagatcacccggaacaaggactacggcctcttctcgtcgcggttcgcgaagcgtcacggcttccacctcctcggcacggcggcgacgtggttcctggtggacatcgcctactacagccagaacctgttccagaaggatatcttcgctagcatccactggatccccaaggcgcgcaccatgagcgcgctcgaggaggtgttccgcatctcccgcgcgcagacgctcatcgcgctctgcggcaccgtgccgggctactggttcaccgtcttcctcatcgacatcctcggccgcttcgccatccagctcctgggcttcgccatgatgaccgtcttcatgctcggcctcgccgtcccgtaccaccactggaccacgtcgggcaaccacatcggcttcgccgtcatgtatggcttcaccttcttcttcgccaacttcgggcccaacgcgacgacgttcatcgtcccggccgagatcttcccggcgcgtctccggtccacctgccacggcatctccgccgctgccggtaaggccggcgcaatcatcggagccttcgggttcctctacgcggcgcagcccaaggacaaggcgcacgtggacgccgggtacaagccagggatcggcgtgcagaacgcgctcatcgtgctcgccgtgtgcaacttcctagggttcttgttcaccttcctggtgccggaatccaaagggaagtcgcttgaggagatgtccggcgaggccaacgaggaggaaaccaccggcaccagcgccaacgccaacgccatgcagccttccggacttgaaatggtgtagacatgcgtacgtgcttttgtgacggtactaggcagagagatctttgttagcacgtaggattattatacatcaattttcttgtactgaacttgaggtgttgaattcgaaatttatttcaaattcttatggaatgtgctgattttttata

SEQ ID NO: 21 - protein, 543 aa, Setaria italica
MARQERRAQLQVLTTLDAAKTQWYHFTAIVVAGMGFFTDAYDLFCISLVTKLLGRIYYTDPASPDPGTLPPNVAAAVNGVALCGTLAGQLFFGWLGDKLGRKSVYGMTLLLMVICSVASGLSFGSTPNGVMATLCFFRFWLGFGIGGDYPLSATIMSEYANKKTRGAFIAAVFAMQGFGILAGGIVTLILSTVFRKAFPAPAYLVDAAASTVPQADYVWRIILMLGAAPAILTYYWRTKMPETARYTALVAKNAKQAAADMSKVLQVEIDAESEKLDEITRNKDYGLFSSRFAKRHGFHLLGTAATWFLVDIAYYSQNLFQKDIFASIHWIPKARTMSALEEVFRISRAQTLIALCGTVPGYWFTVFLIDILGRFAIQLLGFAMMTVFMLGLAVPYHHWTTSGNHIGFAVMYGFTFFFANFGPNATTFIVPAEIFPARLRSTCHGISAAAGKAGAIIGAFGFLYAAQPKDKAHVDAGYKPGIGVQNALIVLAVCNFLGFLFTFLVPESKGKSLEEMSGEANEEETTGTSANANAMQPSGLEMV

SEQ ID NO: 22 - DNA, 3115 bp, Oryza sativa
tcggttcttcagagttacagtgctaacggcctgcagcagagtgcagtgactcccctgaagaaactggtatattaatatcaggtgtgtatatatttcacattttattctagtactactattaatgacatgtctatatatgtcaattttaagtacatacatgtatgggagattaaatttttgatatgttcacaagtttcttgctgatgaaacatgcgtcaaggcaggatgttgtgtaagggtgttaattactgattggtcattagttgccctcatgaatccatgaaaaagttcttcataaagtcatcacaagaagagaccttttgtgctctctttacggcatgcaaaggtcacgaacagtttaacaaaaacacctttgcatactagtctccctcctcgtcatacttcagcaaccacaagatttcttttctgaactttttactaatgaacattcagaaatttctgtgcaatattatctcatgacctgaaccaaacgatgcttgagccacgaaatagtagaggagacaaagatatagtttcgtcaattcgagaagtttgtccggatactacggatgatagcggcagatttggactggttccatgaaagttgtacagtaaggtgcgaatcttgagttgcagagatgcacctggatccggctatctagcttcacgagaatcccatctctactctcctaaattgcccacgaaactgaatttatgtagggatttttagcgaaattcagacatttttcacggggatgggtcggggattgttgactgataaagctggatttgaagaaacaacaaaattttgatatatgataccttgaataaacgaggagtttctgaagtagtggcatggtctgttccagatgtctctctgaacttccgtttcagtttcagtggaccatattgttggtgaactgaaacgaatattatcttctcgtagccacgtgcattctgtagattttcttttgctcagttcgacacatagacatctgaggctaattagctctgttaatcgcgcggtttgtgtaattctcacaaataattagtttctcgttcattgcaaattgcagcgagattttgtcgaaataataaacttggtgttcagttattctctgcaaaaaattgcatattgcagagtagctgagattggcgccatggccggcgagctcaaggtgctgaacgcgctcgactcggcgaagacgcagtggtaccatttcacggcgatcgtgatcgccggcatggggttcttcaccgacgcctacgacctcttctccatctccctcgtcaccaagctgctcggccgcatctactacttcaacccggcgtccaagagccccggctccctcccgcccaacgtctccgccgccgtcaatggcgtcgccttctgcggcaccctcgccggccagctcttcttcggttggctcggcgacaagatggggcgcaagaaggtgtacggcatgacgctcatgctcatggtcatctgctgcctcgcttccggcctctcgttcgggtcgtcggcgaaaggcgtcatggccacgctctgcttcttccgcttctggctcggcttcggcatcggcggcgactacccgctctcggcgaccatcatgtcggagtacgctaataagcgcacccgtggagcgttcatcgccgccgtgttcgccatgcagggcttcggcaacctcaccggcggcatcgtggccatcatcgtgtccgccgcgttcaagtcgcggttcgacgcgccggcgtacagggacgaccggaccggctccaccgtgccgcaggccgactacgcgtggcgcatcgtgctcatgttcggcgccatcccggcgctgctcacctactactggcggatgaagatgccggagacggcgcgctacaccgcgctggtcgccaagaacgcgaagcaggccgccgcggacatgacgcaggtgctcaacgtcgagatcgtggaggagcaggagaaggctgacgaggtcgcgcggcgcgagcagttcgggctcttctcccgccagtttttgagacgccatgggcgccacctgctgggcacgacggtgtgctggttcgtgctggacatcgccttctactcgtcgaacctgttccagaaggacatctacacggcggtgcagtggctgcccaaggcggacaccatgagcgccctggaggagatgttcaagatctcccgggcacagacgctcgtggcgctgtgcggcaccattccgggctactggttcaccgtcttcttcatcgacatcatcggccgcttcgtcatccagctcggcggcttcttcttcatgacggcgttcatgctcggcctcgccgtgccgtaccaccactggacgacgccggggaaccacatcggcttcgtggtcatgtacgccttcaccttcttcttcgccaacttcgggcccaactccacgaccttcatcgtgccggcggagatcttcccggcgaggctgcgttccacctgccacggcatctcggcggcggcggggaaggccggcgccatcgtcgggtcgttcgggttcctgtacgcggcgcagagcacggacgcgagcaagacggacgccggctacccgccgggcatcggcgtgcgcaactcgctcttcttcctcgccggatgcaacgtcatcgggttcttcttcacgttcctggtgccggagtcgaaggggaagtcgctggaggagctctccggcgagaacgaggacgatgacgatgtgccggaagcgcccgcgacggccgatcaccggactgcgccggcgccgccagcttgataccccgcggcaaaacccaaatggtcaatcatcagtgttttgttgtaatatatgtgcaatggatgattattctggttctgctagtgtaccaaacaaaattacaaatactagtcgtcaacccaggcaacgcacgggttactgttgatattataaatgccacttagattatgtattaaatatattttctaaaattattgtggcttaaattttgtaaaaaaagaatattgcggcttagattgcattagaataacaataacatcgcctacaattcacttagtgcccatttgatttggaaaaaaaataaaggaattttggatggttttaatcctacatgaaaa

SEQ ID NO: 23 - protein, 538 aa, Oryza sativa
MAGELKVLNALDSAKTQWYHFTAIVIAGMGFFTDAYDLFSISLVTKLLGRIYYFNPASKSPGSLPPNVSAAVNGVAFCGTLAGQLFFGWLGDKMGRKKVYGMTLMLMVICCLASGLSFGSSAKGVMATLCFFRFWLGFGIGGDYPLSATIMSEYANKRTRGAFIAAVFAMQGFGNLTGGIVAIIVSAAFKARFDAPAYRDDRAGSTVPQADYAWRIVLMFGAIPALLTYYWRMKMPETARYTALVAKNAKQAAADMTQVLNVEIVEEQEKADEVARREQFGLFSRQFLRRHGRHLLGTTVCWFVLDIAFYSSNLFQKDIYTAVQWLPKADTMSALEEMFKISRAQTLVALCGTIPGYWFTVFFIDIIGRFVIQLGGFFFMTAFMLGLAVPYHHWTTPGNHIGFVVMYAFTFFFANFGPNSTTFIVPAEIFPARLRSTCHGISAAAGKAGAIVGSFGFLYAAQSTDASKTDAGYPPGIGVRNSLFFLAGCNVIGFFFTFLVPESKGKSLEELSGENEDDDDVPEAPSTADHRTAPAPPA

SEQ ID NO: 24 - DNA, 1815 bp, Oryza sativa
atggccggcgagctcaaggtgctgaacgcgctcgactcggcgaagacgcagtggtaccatttcacggcgatcgtgatcgccggcatggggttcttcaccgacgcctacgacctcttctccatctccctcgtcaccaagctgctcggccgcatctactacttcaacccggcgtccaagagccccggctccctcccgcccaacgtctccgccgccgtcaatggcgtcgccttctgcggcaccctcgccggccagctcttcttcggttggctcggcgacaagatggggcgcaagaaggtgtacggcatgacgctcatgctcatggtcatctgctgcctcgcttccggcctctcgttcgggtcgtcggcgaaaggcgtcatggccacgctctgcttcttccgcttctggctcggcttcggcatcggcggcgactacccgctctcggcgaccatcatgtcggagtacgctaataagcgcacccgtggagcgttcatcgccgccgtgttcgccatgcagggcttcggcaacctcaccggcggcatcgtggccatcatcgtgtccgccgcgttcaagtcgcggttcgacgcgccggcgtacagggacgaccggaccggctccaccgtgccgcaggccgactacgcgtggcgcatcgtgctcatgttcggcgccatcccggcgctgctcacctactactggcggatgaagatgccggagacggcgcgctacaccgcgctggtcgccaagaacgcgaagcaggccgccgcggacatgacgcaggtgctcaacgtcgagatcgtggaggagcaggagaaggctgacgaggtcgcgcggcgcgagcagttcgggctcttctcccgccagtttttgagacgccatgggcgccacctgctgggcacgacggtgtgctggttcgtgctggacatcgccttctactcgtcgaacctgttccagaaggacatctacacggcggtgcagtggctgcccaaggcggacaccatgagcgccctggaggagatgttcaagatctcccgggcacagacgctcgtggcgctgtgcggcaccattccgggctactggttcaccgtcttcttcatcgacatcatcggccgcttcgtcatccagctcggcggcttcttcttcatgacggcgttcatgctcggcctcgccgtgccgtaccaccactggacgacgccggggaaccacatcggcttcgtggtcatgtacgccttcaccttcttcttcgccaacttcgggcccaactccacgaccttcatcgtgccggcggagatcttcccggcgaggctgcgttccacctgccacggcatctcggcggcggcggggaaggccggcgccatcgtcgggtcgttcgggttcctgtacgcggcgcagagcacggacgcgagcaagacggacgccggctacccgccgggcatcggcgtgcgcaactcgctcttcttcctcgccggatgcaacgtcatcgggttcttcttcacgttcctggtgccggagtcgaaggggaagtcgctggaggagctctccggcgagaacgaggacgatgacgatgtgccggaagcgcccgcgacggccgatcaccggactgcgccggcgccgccagcttgataccccgcggcaaaacccaaatggtcaatcatcagtgttttgttgtaatatatgtgcaatggatgattattctggttctgctagtgtaccaaacaaaattacaaatactagtcgtcaacccaggcaattgatattataaatgccacttagattatgtattaaatatattttctaaaattattgtggcttaaattttgt

SEQ ID NO: 25 - protein, 538 aa, Oryza sativa
MAGELKVLNALDSAKTQWYHFTAIVIAGMGFFTDAYDLFSISLVTKLLGRIYYFNPASKSPGSLPPNVSAAVNGVAFCGTLAGQLFFGWLGDKMGRKKVYGMTLMLMVICCLASGLSFGSSAKGVMATLCFFRFWLGFGIGGDYPLSATIMSEYANKRTRGAFIAAVFAMQGFGNLTGGIVAIIVSAAFKSRFDAPAYRDDRTGSTVPQADYAWRIVLMFGAIPALLTYYWRMKMPETARYTALVAKNAKQAAADMTQVLNVEIVEEQEKADEVARREQFGLFSRQFLRRHGRHLLGTTVCWFVLDIAFYSSNLFQKDIYTAVQWLPKADTMSALEEMFKISRAQTLVALCGTIPGYWFTVFFIDIIGRFVIQLGGFFFMTAFMLGLAVPYHHWTTPGNHIGFVVMYAFTFFFANFGPNSTTFIVPAEIFPARLRSTCHGISAAAGKAGAIVGSFGFLYAAQSTDASKTDAGYPPGIGVRNSLFFLAGCNVIGFFFTFLVPESKGKSLEELSGENEDDDDVPEAPATADHRTAPAPPA

SEQ ID NO: 26 - DNA, 1968 bp, Brachypodium distachyon
gcacaaccaagatgctcgaggcggcggcgaagatcaatccgcggcagccatggcgcggccgcagctggaggtgctgtcgaagctggacgcggcgaagacgcagtggtaccacttcacggcgatcgtgatcgccggcatgggcttcttcacggacgcctacgacctcttctgcatctccctcgtcaccaagctcctgggccgcatctactaccacatcgacggctccccgaccccgggctccctccctcccaacgtcgccgccgccgtcaacggcgtcgccttctgcggcacgctctccggccagctcttcttcggctggctcggcgacaagatgggacgcaagaaggtctacggcatgacgctcatgtgcatggtgctctgctccatcgcctccggcctctccttcggccagacgcccacctccgtcatggccacgctctgcttcttccgcttctggctcggcttcggcatcggcggcgactatccgttgtccgccacgatcatgtcagagtacgccaacaagaagacccggggcgccttcatcgccgccgtcttcgccatgcagggcttcggcatcctgacaggcggcgtggtgactctcgtcatctcctcggcgttcagggcggcgttcgacgcgccggcttacaaggacggcgccatggcgtcgacgccgccccaggccgactacgtgtggcggatcatcctgatgttcggcgccatcccggccctgatgacgtactactggcggatgaagatgccggagacggcgcgctacacggcgcttgtggccaagaacgccaagcaggccgccgccgacatgtccaaggtgctccaggtcgaaatcggcgccgaggaagacaacaacaaggctggcggcgccgtggaggagaaccggaactcgttcgggctgttctcggcggagttcctgcgtcgccacgggcttcacctcctgggcacggccacatgctggttcctgctcgacatcgccttctactcgcagaacctcttccagaaggacatcttcacggccatcaactggatccccaaggcaaacaccatgagcgccctcgaagaagtctaccgcatcgcgcgcgcccagacgctcatcgcgctctgcggcacggtgccgggctactggttcacggtggcgctcatcgacaggatcggcaggttctggatccagctaggggggttcttcttcatgaccgtcttcatgctctgcctggcggcgccgtaccaccactggacgacgcccgggaaccacatcggcttcgtcgtgctctacgggctcaccttcttcttcgcaaacttcgggcccaactccacgacattcatcgtccccgcggagatcttccctgccaggctcaggtccacctgccatggcatctccgctgctgccgggaagctcggcgccattattgggtcctttgggttcctctacctggcgcagagccaggaccccgccaaggtggaccatggctacaaggctgggattggggtcaggaactcgctgtttatcctctctgtttgcaatttcctcgggatgggattcaccttcctcgcgccggagtccaatggcctctcgctcgaggagctctctggggagaacgaagacggcgaggaccagccggcgccggcgcacgccaggacggtgcccgtgtgatggtgaggaatctatacttttattagtgatctgtggtcttgtcttgagttcattagattagagtcggttctattgtgtaaacatgatcaacatgatcgagagttgttaccgagatatgaacaggggatgtgtggtgtgtgggtcagttgcttctacgggcagctagtaatttcgggtgtgtcagtcagtcaggcccaaaatacttactcttttgcagagtttttgctcttaattttgtttctcttctcgtagtactacagcatctccatgatactcgcggaacggagtaatacatacatgctctt

SEQ ID NO: 27 - protein, 537 aa, Brachypodium distachyon
MARPQLEVLSKLDAAKTQWYHFTAIVIAGMGFFTDAYDLFCISLVTKLLGRIYYHIDGSPTPGSLPPNVAAAVNGVAFCGTLSGQLFFGWLGDKMGRKKVYGMTLMCMVLCSIASGLSFGQTPTSVMATLCFFRFWLGFGIGGDYPLSATIMSEYANKKTRGAFIAAVFAMQGFGILTGGVVTLVISSAFRAAFDAPAYKDGAMASTPPQADYVWRIILMFGAIPALMTYYWRMKMPETARYTALVAKNAKQAAADMSKVLQVEIGAEEDNNKAGGAVEENRNSFGLFSAEFLRRHGLHLLGTATCWFLLDIAFYSQNLFQKDIFTAINWIPKANTMSALEEVYRIARAQTLIALCGTVPGYWFTVALIDRIGRFWIQLGGFFFMTVFMLCLAAPYHHWTTPGNHIGFVVLYGLTFFFANFGPNSTTFIVPAEIFPARLRSTCHGISAAAGKLGAIIGSFGFLYLAQSQDPAKVDHGYKAGIGVRNSLFILSVCNFLGMGFTFLAPESNGLSLEELSGENEDGEDQPAPAHARTVPV

SEQ ID NO: 28 - DNA, 1735 bp, Sorghum bicolor
aaccccggttcttctgcctttgcgatctcgcgtaacaaacccccgcaacgcgttggagcgcttggcagcagcagggaattccagcagcagcggcagcagctgctgcacgtcgacatcgcgcggcgccatggcgagggaggagcaggagcggcagcggcagctgcaggtgctgaccacgctcgacgccgccaagacgcagtggtaccacttcacggcgatcgtcgtggcgggcatgggcttcttcaccgacgcctacgacctcttctgcatctcgctcgtcaccaagctgctcggccgcgtctactacaccgaccccaccaagccggacccaggcacgctgccacccaacgtcgcggcggcggtgaacggcgtcgcgctctgcgggacgctcgccgggcagctcttcttcggctggctcggcgacaggctcggccggaagagcgtctacggcatgacgctgctgctcatggtggtctgctccatcgcctcggggctctcgttcgggagcacgccgaccggcgtcatggccacgctctgcttcttccgcttctggctcggtttcggcatcggcggcgactacccgctcagcgccaccatcatgtccgagtacgccaacaagaagacccgcggcgggttcatcgccgccgtcttcgccatgcagggattcggcatcctcggcggcggcatcgtcacgctcgccctctccgcggtgttccgcagggcgtacccggcgccagcgtacctagtcgacgccgtggcgtccaccgtgccgcaggccgactacgtgtggcgcgtcatcctcatgctcggcgcggcgcccgcggtgctgacgtactactggcggaccaagatgcccgagacggcgcggtacaccgcgctggtcgccgggaacgccaagcaggccgcctccgacatgtccagggtgctgcaggtggagatcaaggcggaggcggagaatctggacgagatcaccggaggcagcgcctacggcctcttctcgtcgcggttcgcgcggcgccacggctggcacctcctcggcacggccgtcacgtggttcctggtggacatcgcctactacagccagaacctgttccagaaggacatcttcgccagcatccactggatccccaaggcgcgcaccatgagcgcgctcgacgaggtgttccgcatctcccgcgcgcagacgctcatcgcgctctgcggcaccgtgccgggctactggttcaccgtcttcctcatcgacgtcctcggccgattcgccatccagctcctgggcttcgccatgatgaccgtcttcatgctcggcctcgccatcccgtaccaccactggaccacgccaggcaaccacatcggcttcgccgtcatgtacggcttcaccttcttcttcgcaaacttcgggccgaatgcgaccacgttcatcgtgccggccgagatcttcccggcgcggctccggtccacctgccacggcatctccgctgccgccggcaaggcaggcgcgatcataggagccttcggcttcctctacgcggcgcagtctcaggacaaggcgcacgtggacgccgggtataaacctgggatcggtgtcaggaacgcgctgttcgtgctcgccgcctgcaacttgctggggttcttgttcaccttcctggtgccggaatcgaaagggaaatcgctcgaggagatgtccggcgaagccgatggcgaccaagcctccggcaatggcgccaatgccgtgtag

SEQ ID NO: 29 - protein, 535 aa, Sorghum bicolor
MAREEQERQRQLQVLTTLDAAKTQWYHFTAIVVAGMGFFTDAYDLFCISLVTKLLGRVYYTDPTKPDPGTLPPNVAAAVNGVALCGTLAGQLFFGWLGDRLGRKSVYGMTLLLMVVCSIASGLSFGSTPTGVMATLCFFRFWLGFGIGGDYPLSATIMSEYANKKTRGGFIAAVFAMQGFGILGGGIVTLALSAVFRRAYPAPAYLVDAVASTVPQADYVWRVILMLGAAPAVLTYYWRTKMPETARYTALVAGNAKQAASDMSRVLQVEIKAEAENLDEITGGSAYGLFSSRFARRHGWHLLGTAVTWFLVDIAYYSQNLFQKDIFASIHWIPKARTMSALDEVFRISRAQTLIALCGTVPGYWFTVFLIDVLGRFAIQLLGFAMMTVFMLGLAIPYHHWTTPGNHIGFAVMYGFTFFFANFGPNATTFIVPAEIFPARLRSTCHGISAAAGKAGAIIGAFGFLYAAQSQDKAHVDAGYKPGIGVRNALFVLAACNLLGFLFTFLVPESKGKSLEEMSGEADGDQASGNGANAV

SEQ ID NO: 30 - DNA, 1776 bp, Setaria italica
ggtagctgcaaactgcaaggtgaagaaagagtgattgcctagctagccaactgttgcgtaaatggcccacgatcacaaggtgctcgacgcgcttgacgcggccaagacgcagtggtaccacttcacggcggtgatcatcgccggcatgggcttcttcaccgacgcctacgacctattctccatctccctcgtcaccaagctcctgggccgcatctactacttcaaccccagctcaaagaccccaggctcgctcccgccgaatgtctccgccgccgtcaacggcgtcgccttctgcggcacgctggccgggcagctcttcttcggctggctcggggacaagatggggcgcaagaaggtgtacggcatgacgctcatgctcatggtcatctgctgcctcgcctcgggcctctcgttcgggtcctcgcccaagggcgtcatggccacgctctgcttcttcaggttctggctcggcttcggcatcggcggcgactacccgctctccgcgaccatcatgtccgagtacgccaacaagcggacgcgcggcgcgttcatcgcagccgtcttcgccatgcagggcttcggcaacctcaccggcggcatcgtcgccatcatcatctccgccacgttcaaggcgcgcttcgacgcgccggcgtacaaggacgaccccgccggctccaccgtgccggcggcggactacgcgtggcgcgtcgtcctcatgttcggcgccatcccggcgctgctcacctactactggcgcatgaagatgccggagacggcgcgatacaccgcgctggtcgccaagaacgccaagaaagcgacgtccgacatggcgcgggtgctcaacgtcgagctcaccgaggagcagaagaaggcggaggaggaactcgagcgccgcgaggagtacggcctcttctcccggcagttcgccaagcggcacggcctgcaccttctcggcacgacggtgtgctggttcatgctggacatcgccttctactcgcagaacctattccagaaggacatctacaccgccgtgaactggctgcccaaggcggagaccatgaacgccctcgaggagatgttcaggatctcccgcgcgcagacgctcgtggcgctgtgcggcaccatcccgggctactggttcaccgtcttcttcatcgacatcgtcggccgcttcgccatccagctcggcggcttcttcttcatgacggcgttcatgctcggcctcgccatcccgtaccaccactggacgacgtccgggaaccacgccggcttcgtcgtcatgtacgccttcaccttcttcttcgccaacttcgggcctaactccaccaccttcatcgtgccggcagagatcttcccggcgcggctgcggtccacatgtcacggcatttcctcagctgcaggcaagtccggcgccattgtcgggtcattcgggttcctctacgcggcgcagagcaccgacccggccaagacggatgccggttacccgccaggcatcggcgtgcgcaactcactgttcatgctcgccggatgcaatgtcatcgggttcttgttcacgttccttgtgccggagtccaagggaaagtcgctggaggagctctccggcgagaacgacgaggaggcagcacctggccagagcatccagcagactgttccgaccaatttgagcgaataaatagtatactgtttctatatacttaccaatgtctacacttccgtagggctatatgttagtccatcaggatttatgaacttggttgaattggcatgtttgtaata

SEQ ID NO: 31 - protein, 536 aa, Setaria italica
MAHDHKVLDALDAAKTQWYHFTAVIIAGMGFFTDAYDLFSISLVTKLLGRIYYFNPSSKTPGSLPPNVSAAVNGVAFCGTLAGQLFFGWLGDKMGRKKVYGMTLMLMVICCLASGLSFGSSPKGVMATLCFFRFWLGFGIGGDYPLSATIMSEYANKRTRGAFIAAVFAMQGFGNLTGGIVAIIISATFKARFDAPAYKDDPAGSTVPAADYAWRVVLMFGAIPALLTYYWRMKMPETARYTALVAKNAKKATSDMARVLNVELTEEQKKAEEELERREEYGLFSRQFAKRHGLHLLGTTVCWFMLDIAFYSQNLFQKDIYTAVNWLPKAETMNALEEMFRISRAQTLVALCGTIPGYWFTVFFIDIVGRFAIQLGGFFFMTAFMLGLAIPYHHWTTSGNHAGFVVMYAFTFFFANFGPNSTTFIVPAEIFPARLRSTCHGISSAAGKSGAIVGSFGFLYAAQSTDPAKTDAGYPPGIGVRNSLFMLAGCNVIGFLFTFLVPESKGKSLEELSGENDEEAAPGQSIQQTVPTNLSE

SEQ ID NO: 32 - protein, 539 aa, Theobroma cacao
MAEGQLQVLNALDVAKTQWYHFTAIIIAGMGFFTDAYDLFCISLVTKLLGRIYYHIDGAEKPGTLPPNVSAAVNGVAFCGTLAGQLFFGWLGDKLGRKKVYGMTLMLMVICSIASGLSFGHTPKSVMATLCFFRFWLGFGIGGDYPLSATIMSEYANKKTRGAFIAAVFAMQGFGILAGGIFAIIISSAFKARFDAPPYEVDALGSTVPQADYVWRIILMVGALPAALTYYWRMKMPETARYTALVAKNAKQAASDMSKVLQMDIEAEPQKIEQLDRERSKFGLFSKEFAKRHGFHLLGTTTTWFLLDIAFYSQNLFQKDIFSAIGWIPAAKTMNALDEVFRIARAQTLIALCSTVPGYWFTVAFIDKIGRFSIQLMGFFFMTVFMFALAIPYDHWTHKDNRIGFVVMYSLTFFFANFGPNATTFVVPAEIFPARLRSTCHGISAASGKLGAIVGAFGFLYLAQNKDKAKADAGYPAGIGVKNSLLVLGAINALGFLFTFLVPESKGKSLEEMSGENEDNGAEVEAELSSHNHRTVPVA

SEQ ID NO: 33 - protein, 536 aa, Ricinus communis
MAGPRLEVLNALDIAKTQWYHFTAIVIAGMGFFTDAYDLFCISLVTKLLGRIYYTDYTKDKPGSLPPDVAAAVNGVALCGTLAGQLFFGWLGDKLGRKKVYGITLILMVVCSLASGLSFGSSPKGTIATLCFFRFWLGFGIGGDYPLSATIMSEYANKKTRGAFIAAVFAMQGFGILAGGIVALIVSSAFNNRFPAPTYAVDRRASLIPQADYVWRIILMFGAIPAALTYYWRMKMPETARYTALVAKNAKQAAADMSKVLNVDLEAEEEKVTKIVTEPNNSFGLFSKEFAKRHGLHLVGTTTTWFLLDIAFYSQNLFQKDIFTAINWIPKAAEMNALYEVFRIARAQTLIALCSTVPGYWFTVFLIDYMGRFAIQLMGFFFMTVFMFALAIPYDHWTLKPNRIGFVVMYSLTFFFANFGPNATTFVVPAEIFPARLRSTCHGISAAAGKAGAIVGAFGFLYAAQSQDKTKTDAGYPPGIGVKNSLIALGVINFIGMLFTLLVPESKGRSLEELTGENDESGEEMQAAASVRTVPV

SEQ ID NO: 34 - protein, 541 aa, Camellia oleifera
MAKEQLQVLNALDVAKTQWYHFTAIVIAGMGFFTDAYDLFCISLVTKLLGRIYYHNDGDAKPGSLPPNVSAAVNGVAFCGTLAGQLFFGWLGDKMGRKRVYGMTLMVMVIAAIASGLSFGKSAKGVMTTLCFFRFWLGFGIGGDYPLSATIMSEYANKRTRGAFIAAVFAMQGFGILTGGMVACIISASFKAKFPAPAYQVNPLGSTVPEADYVWRIILMFGAIPAALTYYWRMKMPETARYTALIAKNAKQAAADMSKVLQVELEAEQEKVEKLSEDKGNDFGLFSKQFLHRHGLHLLGTTTTWFLLDIAFYSQNLFQKDIFSAIGWIPDAKTMNAIEEVFRISRAQTLIALCSTVPGYWFTVALIDKIGRFTIQLMGFFFMTVFMYALAIPYNHWTHKENRIGFVVMYSLTFFFANFGPNATTFVVPAEIFPARLRSTCHGISAASGKAGAIVGAFGFLYAAQNQDPTKTDKGYPPGIGVRNALMVLGGVNFLGMVFTFLVPESKGKSLEEMSQENEEDEDGSTEMRQQTSHDIRTVPV

SEQ ID NO: 35 - protein, 537 aa, Nicotiana tabacum
MAKDQLQVLNALDVAKTQLYHFTAIVIAGMGFFTDAYDLFCISLVTKLLGRIYYHHDGAPKPGTLPPNVSAAVNGVAFCGTLAGQLFFGWLGDKMGRKRVYGMTLMMMVICSIASGLSFGHTPKSVMTTLCFFRFWLGFGIGGDYPLSATIMSEYANKKTRGAFIAAVFAMQGFGILAGGMVAIIVSAAFKGAFPAQTYQTDPLGSTVSQADFVWRIILMFGAIPAAMTYYWRMKMPETARYTALVAKNLKQAANDMSKVLQVDIEEEQEKVENVSQNTRNEFGLFSKEFLRRHGLHLLGTASTWFLLDIAFYSQNLFQKDIFSAIGWIPPAQTMNALEEVYKIARAQTLIALCSTVPGYWFTVFFIDKIGRFAIQLMGFFFMTVFMFALAIPYHHWTLKDNRIGFVIMYSLTFFFANFGPNATTFVVPAEIFPARLRSTCHGISAAAGKAGAMIGAFGFLYAAQPTDRKKADAGYPAGIGVRNSLIVLGCVNFLGMVFTFLVPESKGKSLEEMSRENEGEEESGTEMKNSGRTVPV

SEQ ID NO: 36 - protein, 536 aa, Hevea brasiliensis
MAKELQVLSALDVAKTQWYHFTAIIIAGMGFFTDAYDLFCISLVTKLLGRIYYHVDGAEKPGTLPPNVSAAVNGVAFCGTLAGQLFFGWLGDKMGRKKVYGMTLMLMVICSVASGLSFGHNAKAVMSTLCFFRFWLEFGIGGDYPLSATIMSEYANKKTRGAFIAAVFAMQGFGILAGGMFAIIVSSAFRARFDAPAYEVDAVASTVPQADYVWRIILMVGALPAALTYYWRMKMPETARYTALVAKNAKQAASDMSKVLQVDLEAEEQKVQQLAQDKSNSFGLLSKEFLRRHGLHLLGTTSTWFLLDIAFYSQNLFQKDIFSAIGWIPPAKTMNAIEEVFRIARAQTLIALCSTVPGYWFTVAFIDKMGRFAIQLMGFFFMTVFMFALAIPYNHWTHRDNRIGFVVMYSLTFFFANFGPNATTFVVPAEIFPARLRSTCHGISAASGKLGAIVGAFGFLYLAQNKDKAKADAGYPAGIGVRNSLIVLGVVNFLGMVFTLLVPESKGKSLEEMSGENEDDNQPGEQSSYNSRTIAV

SEQ ID NO: 37 - protein, 538 aa, Solanum tuberosum
MANDLQVLNALDVAKTQLYHFTAIVIAGMGFFTDAYDLFCISMVTKLLGRIYYHHDNALKPGSLPPNVSAAVNGVAFCGTLAGQLFFGWLGDKMGRKKVYGMTLMIMVICSIASGLSFGHTPKSVMTTLCFFRFWLGFGIGGDYPLSATIMSEYANKKTRGAFIAAVFAMQGFGILAGGMVAIIVSSAFKGAFPAPAYEVDALASTVSQADFVWRIILMFGAIPAGLTYYWRMKMPETARYTALVAKNLKQAANDMSKVLQVEIEAEPEKVAAISVANGANEFGLFSKEFLRRHGLHLLGTASTWFLLDIAFYSQNLFQKDIFSAIGWIPPAQTMNALEEVYKIARAQTLIALCSTVPGYWFTVAFIDRIGRFAIQLMGFFFMTVFMFALALPYHHWTLKDNRIGFVVMYSLTFFFANFGPNATTFVVPAEIFPARLRSTCHGISAAAGKAGAMVGAFGFLYAAQPTDPKKTDAGYPAGIGVRNSLIVLGCVNFLGMLFTFLVPESKGKSLEEMSRENEGEEETVAEMRATSGRTVPV

SEQ ID NO: 38 - protein, 534 aa, Arabidopsis lyrata
MAKDQLQVLNALDVAKTQWYHFTAIIIAGMGFFTDAYDLFCISLVTKLLGRIYYHVDGAEKPGTLPPNVAAAVNGVAFCGTLAGQLFFGWLGDKLGRKKVYGMTLMVMVLCSVASGLSFGHEPKAVMATLCFFRFWLGFGIGGDYPLSATIMSEYANKKTRGAFVSAVFAMQGFGIMAGGIFAIIISSAFEAKFPAPAYADDALGSTVPQADLVWRIILMVGAIPAAMTYYSRSKMPETARYTALVAKDAKQAASDMSKVLQMEIEPEQQKVDEISKEKSKAFSLFSKEFMSRHGLHLLGTTSTWFLLDIAFYSQNLFQKDIFSAIGWIPPAKSMNAIQEVFKIARAQTLIALCSTVPGYWFTVAFIDVIGRFAIQMMGFFFMTVFMFALAIPYNHWTHKENRIGFVIMYSLTFFFANFGPNATTFVVPAEIFPARFRSTCHGISAASGKLGAMVGAFGFLYLAQSPDKNKTDAGYPPGIGVRNSLIVLGVVNFLGILFTFLVPESKGKSLEEMSGENEDNESSISDNRTVPIV

SEQ ID NO: 39 - DNA, 1578 bp, Triticum aestivum
atggcgactgaacagctcaacgtgttgaaagcactggacgttgccaagacgcagctgtaccatttcaaggcggtcgtgatcgccggcatgggcttcttcacggacgcctacgacctcttctgcatcgccctcgtcaccaagctgctggggcgcatctactacaccgaccctgcccttaacgagcccggccacctcccggcaaacgtgtcggccgccgtgaacggcgtggccctatgcggcacacttgccggccagctcttcttcggctggctcggtgacaagctcggccgcaagagcgtctacggcttcacgctcatcctcatggtcctctgctccatcgcgtccgggctatcgtttggacacgaggccaagggtgtaatggggacgctatgtttcttccgtttctggctcggcttcggtgtcggcggcgactaccctctgagcgccaccatcatgtcggagtatgctaacaagaagacccgcggcacctttatcgccgccgtgtttgccatgcaggggtttggcatcctatttggtactatcgtcacgatcatcgtctcgtccgcattccgacatgcattccctgcaccgccattctacatcgacgccgcggcgtccattggcccggaggccgactatgtgtggcgcatcatcgtcatgttcggcaccatcccggctgccctgacctactactggcgcatgaagatgcccgaaactgcgcggtacacagcactcatcgccggcaacacaaagcaagccacatcagacatgtccaaggtgctcaacaaggagatctcagaggagaacgtccagggtgagcgggccaccggtgatacctggggcctcttctcccgacagttcatgaagcgccacggggtgcacttgctagcgaccacaagcacttggttcctactcgatgtggccttctacagccagaacctgttccagaaggacatcttcaccaagatcgggtggatcccgccggccaagactatgaatgcattggaggagttgtaccgcatcgcccgcgcccaagcgctcatcgcgctctgtggcaccgtgcctggctactggttcaccgtcgccttcatcgacatcattgggaggttttggatccagctcatgggattcaccatgatgaccattttcatgctagcaatcgccataccgtacgactacttggtggagccagggcatcacaccggctttgtcgtgctctacgggctcactttcttcttcgccaacttcggccccaacagcacaaccttcatcgtgccagctgagatcttccctgcgaggctccgatccacatgccacggtatctctgctgctaccggtaaggcaggcgcgatcatcggtgcattcgggttcctgtatgcgtcacaggaccagaagaagcctgagaccggctactcacggggaatcggcatgcgcaacgctctcttcgtgctcgcgggcacaaacttcctgggtctgctcttttcgctgctggtgccggagtccaagggcaagtcgctggaggagctctccaaggagaacgttggcgacgatgactccattgccccaactggtgtctag

SEQ ID NO: 40 - protein, 525 aa, Triticum aestivum
MATEQLNVLKALDVAKTQLYHFKAVVIAGMGFFTDAYDLFCIALVTKLLGRIYYTDPALNEPGHLPANVSAAVNGVALCGTLAGQLFFGWLGDKLGRKSVYGFTLILMVLCSIASGLSFGHEAKGVMGTLCFFRFWLGFGVGGDYPLSATIMSEYANKKTRGTFIAAVFAMQGFGILFGTIVTIIVSSAFRHAFPAPPFYIDAAASIGPEADYVWRIIVMFGTIPAALTYYWRMKMPETARYTALIAGNTKQATSDMSKVLNKEISEENVQGERATGDTWGLFSRQFMKRHGVHLLATTSTWFLLDVAFYSQNLFQKDIFTKIGWIPPAKTMNALEELYRIARAQALIALCGTVPGYWFTVAFIDIIGRFWIQLMGFTMMTIFMLAIAIPYDYLVEPGHHTGFVVLYGLTFFFANFGPNSTTFIVPAEIFPARLRSTCHGISAATGKAGAIIGAFGFLYASQDQKKPETGYSRGIGMRNALFVLAGTNFLGLLFSLLVPESKGKSLEELSKENVGDDDSIAPTGV